SEQUENCE LISTING (52 sequences)

SEQ ID NO: 1 - DNA, 1750 bp, Artificial Sequence
  Description of Artificial Sequence/note = Synthetic Construct
atacataatcgactattcccctacctgctacttttcccttacaacacgtggattaccatactcttcctctttcccctccagcctgcttttcctctaaatattgaaaccctcaaattcatctttggagaaagtcacagaccacatactgtttctgtgattctgtgttgttttcttccaggcatgtccttaaccttggcaaaataaacttctaaattaattgccacctgtctcagatacccttggttttacagtaagaattcacatgcttacatagtaactattaattttggtttcttagtgtgttacctatatattgtatgcctttgtttctaatgatatttataaagtattgatgccagctcattgttatcacattgccagttaaaaaaaaaactaatttttaacctaaaaattagataagagctggagatggatctgggggtagaacgggggcagtagtaaaagcacgcgtcagagtgggtggggctgtcgaaggtttctgaagaccacagagtgtgataacaaggttttacagtgtcatttccttaagtattaaagtattacagcctgcttcctgagaaggcatttgggaaagagtccccagaaaggggccccaggagagattccagagaaactcgtgttttggagatgttgagggtgaacaaatggtaatgaggatgatgacagaacgcaagaaaagagaactagcattcaccggacacccacgatgtaccaagcactttgctaaccctcatattctcttttactttccaaaaacctgtagtactgtggttctcagccaggggcaattcatcccctggggaacacttgctaatatctgggggcatatttgttttcacaactgggagtgccactggcatctaacaggtagagcccaggagtgctgcggaacatcctacaatgctcagggaaggtcctcacaagaataatttggcccacaatgtccatagtgctcaggctgagaaaccgtgcttaaatggtaggcacaataatcttcacttttacagattggaacctgacactctgagaagccacctggcattcaacccgaaacctaacacagctccaaagcccatgctctttcaccatgccgttgcagtgagaacagggatggaaatgagggtggcaaaaatgaccaagatacaaaaccagggcacagattggtgctcaatagatacttattgggatattcattaaatagaagaatgaataagaaaaagaatgaatgagggcaggggaataatgaggatgagtgtggtcattctattgccatcctgacatacctccttgtccttgttccacaactcagcagtgagtctgggattatgacaatagagaaaattaaatgatggtaggtggcctggagtccccatgctcaatttcaagaagcatccagattccagggcctgggtctccaaatggaagtagaagtactagaagattgctggtgcacgctgtcctgcatcaccctttctcaggaggatagagactgaaacaggaggttctgagctgagttttggtgaccatttccctctttctcccagaggcccaggccagctgtggcctcagaggaagaagaagggagttgtttccctagtttctaaaatttctgtgaatttgaacatgggctacaccagatttattctgggaagctctgaatcttctaggagggaaagactgagaggaaagagggtggaaagggaggagcctgtgataaaacagaacattt

SEQ ID NO: 2 - DNA, 11 bp, Artificial Sequence
  Description of Artificial Sequence/note = Synthetic Construct
aagacaataca

SEQ ID NO: 3 - DNA, 11 bp, Artificial Sequence
  Description of Artificial Sequence/note = Synthetic Construct
aagacantaca

SEQ ID NO: 4 - DNA, 11 bp, Artificial Sequence
  Description of Artificial Sequence/note = Synthetic Construct
aagacaanaca

SEQ ID NO: 5 - DNA, 9 bp, Artificial Sequence
  Description of Artificial Sequence/note = Synthetic Construct
gttgttttc

SEQ ID NO: 6 - DNA, 9 bp, Artificial Sequence
  Description of Artificial Sequence/note = Synthetic Construct
gttgtnttc

SEQ ID NO: 7 - DNA, 10 bp, Artificial Sequence
  Description of Artificial Sequence/note = Synthetic Construct
acagtaagaa

SEQ ID NO: 8 - DNA, 10 bp, Artificial Sequence
  Description of Artificial Sequence/note = Synthetic Construct
acagtaanaa

SEQ ID NO: 9 - DNA, 10 bp, Artificial Sequence
  Description of Artificial Sequence/note = Synthetic Construct
aagagctgga

SEQ ID NO: 10 - DNA, 10 bp, Artificial Sequence
  Description of Artificial Sequence/note = Synthetic Construct
aagagctnga

SEQ ID NO: 11 - DNA, 10 bp, Artificial Sequence
  Description of Artificial Sequence/note = Synthetic Construct
tgttttggag

SEQ ID NO: 12 - DNA, 10 bp, Artificial Sequence
  Description of Artificial Sequence/note = Synthetic Construct
tgttttgnag

SEQ ID NO: 13 - DNA, 9 bp, Artificial Sequence
  Description of Artificial Sequence/note = Synthetic Construct
attcaccgg

SEQ ID NO: 14 - DNA, 9 bp, Artificial Sequence
  Description of Artificial Sequence/note = Synthetic Construct
attcacngg

SEQ ID NO: 15 - DNA, 10 bp, Artificial Sequence
  Description of Artificial Sequence/note = Synthetic Construct
tagtgctcag

SEQ ID NO: 16 - DNA, 10 bp, Artificial Sequence
  Description of Artificial Sequence/note = Synthetic Construct
tagtgctnag

SEQ ID NO: 17 - DNA, 10 bp, Artificial Sequence
  Description of Artificial Sequence/note = Synthetic Construct
ctgtcctgca

SEQ ID NO: 18 - DNA, 10 bp, Artificial Sequence
  Description of Artificial Sequence/note = Synthetic Construct
ctgtcctnca

SEQ ID NO: 19 - DNA, 10 bp, Artificial Sequence
  Description of Artificial Sequence/note = Synthetic Construct
acatttcttt

SEQ ID NO: 20 - DNA, 10 bp, Artificial Sequence
  Description of Artificial Sequence/note = Synthetic Construct
acatntcttt

SEQ ID NO: 21 - DNA, 22 bp, Artificial Sequence
  Description of Artificial Sequence/note = Synthetic Construct
aaagagggtggaaagggaggag

SEQ ID NO: 22 - DNA, 24 bp, Artificial Sequence
  Description of Artificial Sequence/note = Synthetic Construct
ctctcaaagcttggcggattctac

SEQ ID NO: 23 - DNA, 17 bp, Artificial Sequence
  Description of Artificial Sequence/note = Synthetic Construct
tcaagaagcatccagat

SEQ ID NO: 24 - DNA, 24 bp, Artificial Sequence
  Description of Artificial Sequence/note = Synthetic Construct
aaactcagctcagaacctcctgtt

SEQ ID NO: 25 - DNA, 33 bp, Artificial Sequence
  Description of Artificial Sequence/note = Synthetic Construct
ctccacaggttactcgtttctaccttatcttac

SEQ ID NO: 26 - DNA, 23 bp, Artificial Sequence
  Description of Artificial Sequence/note = Synthetic Construct
gcttgcgtggcccctggttctca

SEQ ID NO: 27 - DNA, 25 bp, Artificial Sequence
  Description of Artificial Sequence/note = Synthetic Construct
gttactcgtttctaccttatcttac

SEQ ID NO: 28 - DNA, 23 bp, Artificial Sequence
  Description of Artificial Sequence/note = Synthetic Construct
ttgcagtcagcccagtcactctc

SEQ ID NO: 29 - DNA, 22 bp, Artificial Sequence
  Description of Artificial Sequence/note = Synthetic Construct
atttcaagaagcatccagattc

SEQ ID NO: 30 - DNA, 22 bp, Artificial Sequence
  Description of Artificial Sequence/note = Synthetic Construct
aaagagggtggaaagggaggag

SEQ ID NO: 31 - DNA, 24 bp, Artificial Sequence
  Description of Artificial Sequence/note = Synthetic Construct
ctctcaaagcttggcggattctac

SEQ ID NO: 32 - DNA, 22 bp, Artificial Sequence
  Description of Artificial Sequence/note = Synthetic Construct
tcaagaagcatccagattccag

SEQ ID NO: 33 - DNA, 24 bp, Artificial Sequence
  Description of Artificial Sequence/note = Synthetic Construct
aaactcagctcagaacctcctgtt

SEQ ID NO: 34 - DNA, 20 bp, Artificial Sequence
  Description of Artificial Sequence/note = Synthetic Construct
cctgtgataaaacagaacat

SEQ ID NO: 35 - DNA, 19 bp, Artificial Sequence
  Description of Artificial Sequence/note = Synthetic Construct
tgctggtgcacgctgtcct

SEQ ID NO: 36 - DNA, 31 bp, Artificial Sequence
  Description of Artificial Sequence/note = Synthetic Construct
gcaggtacccatgtatcagagcttggccatg

SEQ ID NO: 37 - DNA, 32 bp, Artificial Sequence
  Description of Artificial Sequence/note = Synthetic Construct
gaagaattcagattacgcagtgattatgtccc

SEQ ID NO: 38 - DNA, 30 bp, Artificial Sequence
  Description of Artificial Sequence/note = Synthetic Construct
cgcggatccaccatggcctcgggcgacacc

SEQ ID NO: 39 - DNA, 31 bp, Artificial Sequence
  Description of Artificial Sequence/note = Synthetic Construct
cggaattctcactggttgtttttggccttag

SEQ ID NO: 40 - DNA, 32 bp, Artificial Sequence
  Description of Artificial Sequence/note = Synthetic Construct
tcggaattcatggctatggagacccaaatgtc

SEQ ID NO: 41 - DNA, 34 bp, Artificial Sequence
  Description of Artificial Sequence/note = Synthetic Construct
ctgtctagattagttattactgttgacatggtcg

SEQ ID NO: 42 - DNA, 16 bp, Artificial Sequence
  Description of Artificial Sequence/note = Synthetic Construct
acatgccnggggggct

SEQ ID NO: 43 - DNA, 15 bp, Artificial Sequence
  Description of Artificial Sequence/note = Synthetic Construct
ttctcccntttggat

SEQ ID NO: 44 - DNA, 15 bp, Artificial Sequence
  Description of Artificial Sequence/note = Synthetic Construct
gagagccncatctca

SEQ ID NO: 45 - DNA, 15 bp, Artificial Sequence
  Description of Artificial Sequence/note = Synthetic Construct
gggcttnttgggagt

SEQ ID NO: 46 - DNA, 15 bp, Artificial Sequence
  Description of Artificial Sequence/note = Synthetic Construct
aagacanacattcga

SEQ ID NO: 47 - DNA, 9 bp, Artificial Sequence
  Description of Artificial Sequence/note = Synthetic Construct
gggantgct

SEQ ID NO: 48 - DNA, 9 bp, Artificial Sequence
  Description of Artificial Sequence/note = Synthetic Construct
ggganacct

SEQ ID NO: 49 - DNA, 9 bp, Artificial Sequence
  Description of Artificial Sequence/note = Synthetic Construct
cggggnact

SEQ ID NO: 50 - DNA, 9 bp, Artificial Sequence
  Description of Artificial Sequence/note = Synthetic Construct
tacacntgc

SEQ ID NO: 51 - DNA, 9 bp, Artificial Sequence
  Description of Artificial Sequence/note = Synthetic Construct
acgctnttc

SEQ ID NO: 52 - DNA, 9 bp, Artificial Sequence
  Description of Artificial Sequence/note = Synthetic Construct
ctcncagga

(19) United States
(12) Patent Application Publication
(10) Pub. No.: US 2008/0248465 A1
(43) Pub. Date: Oct. 9, 2008

(54) Polymorphisms in the Fcgr2b Promoter and Uses Thereof

(75) Inventors: Robert P. Kimberly, Birmingham, AL (US); Jeffrey Edberg, Vestavia, AL (US); Kaihong Su, Vestavia, AL (US); Jianming Wu, Vestavia, AL (US)

Correspondence Address: Ballard Spahr Andrews & Ingersoll, LLP, SUITE 1000, 999 PEACHTREE STREET, ATLANTA, GA, 30309-3915, US

(73) Assignee: UAB Research Foundation, Birmingham, AL (US)

(21) Appl. No.: 11/587,781

(22) PCT Filed: Apr. 26, 2005
(86) PCT No.: PCT/US2005/014531

§ 371 Date: Jan. 17, 2007

Related U.S. Application Data
(60) Provisional application No. 60/565,314, filed on Apr. 26, 2004.

(51) Int. Cl. C12Q 1/68; C07H 21/00; G01N 33/68; G01N 33/00

(52) U.S. Cl. 435/6; 536/22.1; 436/94; 436/86

(57) ABSTRACT

The invention relates to the FCGR2B gene and its promoter. In particular, the invention relates to FCGR2B promoters with specific nucleotides at polymorphic sites. Characterization of the nucleotides at polymorphic sites is useful for characterizing the gene and the protein and is useful for determining predisposition or susceptibility to certain diseases and infections in a subject or a population of subjects. Such characterization of the gene or protein is also useful for determining immunoresponsiveness or responsiveness to therapeutic agents in a subject or population of subjects. Thus, disclosed herein are a variety of related nucleic acids, methods and tools.

### CROSS REFERENCE TO RELATED APPLICATIONS

The application claims the benefit of U.S. Provisional Application Ser. No. 60/565314, filed Apr. 26, 2004, which is herein incorporated by this reference in its entirety.

This invention was made with government support under grants NIH P50 AR45231, NIH P01 AR49084 and NIH R01 AR42476 awarded by the National Institutes of Health. The government has certain rights in the invention.

### FIELD OF THE INVENTION

The present invention relates to FCGR2B promoter polymorphisms and the association of FCGR2B promoter polymorphisms with inflammatory disease, infection, ability to mount an immune response, and responsiveness to therapeutic agents.

### BACKGROUND

FcγRIIb, the immuno-receptor tyrosine-based inhibitory motif (ITIM)-containing receptor for immunoglobulin G, (MIM 604590) plays an important role in maintaining the homeostasis of immune responses. Within the classical IgG Fc-binding receptor family, FcγRIIb (CD32B) is the only receptor that bears an ITIM in its cytoplasmic domain (1) FcγRIIb is expressed on B lymphocytes, myeloid cell lineages, dendritic and mast cells. On B lymphocytes, co-ligation of FcγRIIb with the B cell antigen receptor (BCR) by IgG immune complexes downregulates BCR signaling and modulates the threshold for B cell activation and proliferation (2-6). Co-ligation of FcγRIIb also provides a negative feedback mechanism for immunoglobulin (Ig) production by B cells. On myeloid lineage cells, FcγRIIb co-clustering with the activating Fcγ receptors, such as FcγRIa (CD64), FcγRIIa (CD32A), and FcγRIIIa (CD16A), down-modulates their function (2). Antibody-mediated phagocytosis by macrophages is decreased by exaggerated FcγRIIb co-clustering and is enhanced by disruption of FcγRIIb (7-9). On follicular dendritic cells (FDC), FcγRIIb mediates the retention and conversion of immune complexes to a highly immunogenic form, which facilitate B cell recall responses (10-13). Thus, FcγRIIb plays multiple roles in modulating immune function and thus maintaining immune homeostasis. Indeed, studies in mouse models have highlighted the role of FCGR2B in the development of autoimmune diseases (14-19). For example, targeted disruption of FCGR2B in the mouse leads to elevated serum Ig levels and, on the susceptible C57BL/6 background, leads to the development of lupus-like phenotypes (20, 21).

Human SLE is a prototypic autoimmune disease characterized by production of antinuclear autoantibodies and tissue deposition of immune complexes (22-25). This complex polygenic disease has strong genetic components (λs≈20) (26, 27). In humans, outside of MHC class II, genetic polymorphisms or defects in genes involved in antigen uptake, processing and immune complex clearance such as complement, FCGR2A and FCGR3A have been identified to contribute to SLE susceptibility (26, 28-33). Recently, programmed cell death gene 1 (PDCD1) which regulates B cell activation has been identified as an autoimmunity candidate gene in the mouse (34, 35), and a single nucleotide polymorphism in a putative RUNX1 binding site in the promoter of human PDCD1 gene has been implicated as a risk allele for SLE (34, 35). However, potential variations in the regulatory regions of human FCGR2B as a disease susceptibility gene have not yet been characterized.

### BRIEF DESCRIPTION OF THE FIGURES

FIG. 1 shows SNPs in the 2 kb FCGR2B promoter region. The polymorphic alleles are indicated in the parentheses with the common allele in the upper left and the uncommon allele in the lower right. The nucleotide position is relative to the translation start site.

FIG. 2 shows 5′-deletion analysis of the FCGR2B promoter. A series of 5′-deletion FCGR2B promoter fragments was placed in front of the firefly luciferase report gene and the plasmid was co-transfected with the reference plasmid pRL-SV40 (SV40 promoter drives renilla luciferase gene) into BJAB cells. Dual luciferase assay was performed 24-40 hours after transfection. The firefly luciferase activity was normalized by renilla luciferase levels and the ratio is designated as relative luciferase activity (RLA). The results represent the mean±SEM from 3 independent experiments.

FIG. 3 shows that the variant −386C-120A haplotype of FCGR2B promoter drives higher luciferase reporter expression than the −386G-120T haplotype. The reporter constructs incorporating the four haplotypes (“CGT, GGT, CCA and GCA” are shortened haplotype names and represent alleles at nt −893, −386, and −120 respectively) in the context of 1.0 kb of the FCGR2B promoter were transiently transfected into BJAB (A) and U937 (B) cells. For panel C and D, the reporter constructs with the CGT or CCA haplotype in the context of the 1 kb FCGR2B promoter were transfected into BJAB (C) or U937 (D) cells for 16 hours and then either unstimulated (open bars), stimulated with 0.5 mM dibutyryl-cAMP (hatched bars) or 400 U/ml of IFN-gamma (dot-filled bars) for additional 24 hours. The firefly luciferase activity was measured and normalized by renilla luciferase levels to yield relative luciferase activity (RLA). The results represent the mean±SEM from 3 independent experiments.

FIG. 4 shows four proximal promoter haplotypes and their frequency in FCGR2B and FCGR2C genes. The four haplotypes (2B.1-4) have different allele combination at nt −386 and −120 but the same “C” allele at nt −893. The FCGR2B and FCGR2C genes have distinct haplotype frequencies.

FIG. 5 shows the −120A allele has increased binding capacity for transcription factor GATA4. A, The sequence of the probes used in the EMSAs. Polymorphic alleles are presented in bold lower case and mutant sites are indicated in bold capital case. Arrows indicate GATA-binding motifs. The GATA-binding probe, “gGATA” is derived from the human A γ-globin gene promoter (21). B, EMSAs were performed with nuclear extracts (NE) from U937, BJAB cells or Cos-7 transfectants and ³²P-radiolabeled −120T and −120A probes. 200-fold unlabelled probe (“NS”: non-specific probes) or 4 μg of antibodies were added to the reaction as indicated.

FIG. 6 shows the −386C allele has increased binding capacity for transcription factor YY1. A, The sequence of the probes used in the EMSAs. Polymorphic alleles are presented in bold lower case and mutant sites are indicated in bold capital case. Arrows indicate YY1-binding motif. The YY1-binding probe “YY1” is derived from the human gp91^{phox} gene promoter (22). B, EMSAs were performed with nuclear extracts (NE) from U937 cells or Cos-7 transfectants and ³²P-radiolabeled −386G and −386C probes. 200-fold unlabelled probe or 4 μg of antibodies were added to the reaction as indicated.

FIG. 7 shows that YY1 and GATA4 are expressed in BJAB and U937 cells. Gene-specific RT-PCR for YY1 and 6 GATA family members were performed from RNA prepared from BJAB, U937 cell lines and primary tonsil cells. The PCR specificity was confirmed by directly sequencing of the PCR products.

FIG. 8 shows over expression of GATA4 and/or YY1 transcription factors leads to increased FCGR2B promoter activity. The FCGR2B promoter reporter constructs pGL-2B.1 or pGL-2B.4 were co-transfected with the reference plasmid pRL-SV40 (SV40 promoter drives renilla luciferase gene) and the GATA4 and/or YY1 expression vector pcDNA3 into BJAB or U937 cells. Dual luciferase assay was performed 40 hours after transfection. The firefly luciferase activity was normalized by renilla luciferase levels and the ratio is designated as relative luciferase activity (RLA). The results represent the mean±SEM from 3 independent experiments (p<0.0001 by ANOVA).

FIG. 9 shows that Haplotype 2B.4 leads to higher expression of endogenous FcγRIIb on EBV-transformed and peripheral blood B lymphocytes. A, The EBV-B cells derived from 2B.1 homozygous (thin gray line), 2B.1/2B.4 heterozygous (thick black line) and 2B.4 homozygous (thick gray line) donors were stained with mIgG1 isotype control (dotted line) or mAb AT-10, followed by staining with FITC-conjugated goat anti-mouse IgG. The binding of the isotype control to the three cell lines was identical and only 1 is shown for clarity. No binding of mAb IV.3 above the isotype control was observed on any line. B, A summary of FcγRIIb expression levels on EBV-transformed cells derived from 18 2B.1 homozygous (open bar), 17 2B.1/2B.4 heterozygous (black solid bar), and one 2B.4 homozygous (gray solid bar) donors by flow cytometry using mAb AT10. (* P<0.0155, Kruskal-Wallis test; MFI: mean fluorescence intensity). The average MFI of the isotype control among the three groups was not different (2B.1/2B.1: 3.6±0.2, n=18; 2B.1/2B.4: 3.6±0.3, n=17; 2B.4/2B.4: 3.9, n=1). C, Whole cell lysate was prepared from Cos-7 cells transiently transfected with FcγRIIa or A20-IIA1.6 cells stably transfected with FcγRIIb and immunoprecipitated with mAb 32.2 (as a negative control), IV.3 or AT-10 and subjected to western blot analysis using rabbit anti-FcγRIIb sera (panel I) or goat anti-FcγRIIa/c antibodies (panel II). D, Whole cell lysate from EBV transformed cells derived from 4 2B.1 homozygous donors (lanes 1-4) and 4 2B.4-containing donors (lanes 5-8, 3 2B.1/2B.4 heterozygous and 1 2B.4 homozygous donors) was subjected to western blot analysis using rabbit anti-FcγRIIb cytoplasmic domain antibody (panel I). The membrane was stripped and re-probed with anti-Lyn antibody as a protein loading control (panel II). E, Whole blood from a 2B.1 homozygous (thin gray line) and a 2B.1/2B.4 (thick black line) normal donor was stained with mAb AT-10-FITC and anti-CD19-APC (for B lymphocytes) antibodies and analyzed by flow cytometry. The binding of the isotype control to the two cell lines was identical and only 1 is shown for clarity. No binding of mAb IV.3 above the isotype control was observed on any line. F, A summary of the expression levels of FcγRIIb on peripheral B-lymphocytes from 12 2B.1 homozygous (open bar) and 8 2B.1/2B.4 heterozygous (solid bar) normal donors (** P<0.0003). The average MFI of the isotype control between the two groups was not different (2B.1/2B.1: 4.1±0.3, n=12; 2B.1/2B.4: 3.9±0.4, n=8).

FIG. 10 shows that haplotype 2B.4 leads to higher expression of FcγRIIb on peripheral CD14⁺ monocytes. CD14⁺ monocytes were purified from whole blood from 4 2B.1/2B.1 (lanes 1-4) and 4 2B.1/2B.4 (lanes 5-8) normal donors. Whole cell lysate prepared from those monocytes was subjected to western blot analysis using specific rabbit anti-FcγRIIb sera (panel A) followed by an anti-Lyn antibody as a protein loading control (panel B).

FIG. 11 shows that the FcγRIIb from 2B.4-containing donors has higher inhibitory effects on BCR-induced Ca²⁺ influx. A, EBV-transformed cells from genotyped donors were stimulated with either goat IgG anti-human K (thick line) or goat F(ab)′₂ anti-human K (thin line). The relative inhibition of BCR-induced Ca²⁺ influx is presented as the ratio of the [Ca²⁺]ᵢ change induced by engagement of BCR alone and the [Ca²⁺]ᵢ change induced by co-engagement of both BCR and FcγRIIb. B, A summary of the relative inhibition of BCR-induced Ca²⁺ influx by FcγRIIb from 3 2B.1 homozygous (open bar) and 3 2B.4-containing donors (solid bar, one 2B.4 homozygous and two 2B.1/2B.4 heterozygous donors; * P<0.0055; results represent the mean±SEM from 3 experiments).

FIG. 12 shows that that the FcγRIIb from 2B.4-containing donors has higher inhibitory effects on anti-BCR induced decrease in cell viability. EBV cells from 5 2B.1 homozygous (open bar) and 5 2B.4-containing donors (solid bar, one 2B.4 homozygous and four 2B.1/2B.4 heterozygous donors) were untreated or stimulated with goat F(ab)′₂ anti-human IgM or goat IgG anti-human IgM for 60 hours. The relative inhibition of anti-BCR mediated decrease in cell viability is presented as the ratio of the ATP levels by engagement of BCR alone and co-engagement of both BCR and FcγRIIb (* P<0.023).

### DETAILED DESCRIPTION OF THE INVENTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific nucleic acids or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a nucleic acid” includes mixtures of nucleic acids, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. The term “subject” includes domesticated animals, such as cats, dogs, etc., livestock (e.g., cattle, horses, pigs, sheep, goats, etc.), and laboratory animals (e.g., mouse, rabbit, rat, guinea pig, etc.). The present invention provides the identification of 10 novel single nucleotide polymorphisms (SNPs) in the promoter region of human FCGR2B gene and the characterization of two functionally distinct haplotypes in its proximal promoter. In luciferase reporter assays, the less frequent promoter haplotype leads to increased expression of the reporter gene in both B lymphoid and myeloid cell lines under constitutive and stimulated conditions. Four independent genome wide scans support linkage of the human Fcγ receptor region to the systemic lupus erythematosus (SLE, OMIM 152700) phenotype. A case-control study in 600 Caucasians indicates a significant association of the less frequent FCGR2B promoter haplotype with the SLE phenotype (odds ratio=1.65, P=0.0054). The FCGR2B haplotype has no linkage disequilibrium with previously identified FCGR2A and FCGR3A polymorphisms and after adjustment for FCGR2A and FCGR3A, FCGR2B showed a persistent association with SLE (odds ratio=1.72, P=0.0083). These results show that an expression variant of FCGR2B can be a risk factor for human lupus and implicate FCGR2B in disease pathogenesis.

The present invention relates to polymorphisms of the FCGR2B promoter region and the use of such polymorphisms to assess their effect on FCGR2B levels, FCGR2B activity and on disease states (e.g., autoimmune disorders and cancer). Unlike the single nucleotide polymorphisms (SNPs) in human FCGR2A and FCGR3A which affect the ligand-binding properties of the receptors (29, 36), no non-synonymous SNPs encoding the extracellular domains of FcγRIIb in more than 120 donors were found in the studies presented herein. However, 10 polymorphic sites were identified in the 2 kb promoter region of human FCGR2B which defined two SNP haplotypes in its proximal promoter. In luciferase reporter assays, the less frequent variant FCGR2B haplotype increases the promoter activity both constitutively and under inducible conditions. In a case-control study of 600 Caucasians, the variant FCGR2B haplotype is significantly associated with the SLE phenotype. This association is not due to the effects of previously identified FCGR2A or FCGR3A polymorphisms. This observation not only provides evidence for the genetic association of FCGR2B with human lupus but also is the first study to characterize the functionally important promoter polymorphisms in FCGR2B, one of the key regulators in immune responses.

The present invention provides specific sites in the FCGR2B gene sequence that are polymorphic, i.e., the nucleotide at a specific position or at specific positions varies across a population of subjects such that the nucleotide can be a G, A, T, C, or a subset thereof at the specific position or positions. Therefore, as utilized herein, the term “polymorphic” or “polymorphic site” means that, at one or more specific positions in a FCGR2B gene promoter nucleotide sequence, the most commonly found nucleotide or another nucleotide that differs from the most commonly found nucleotide can be identified at the specific site across a population of subjects. Therefore, the term “polymorphic” or “polymorphism” encompasses both the most commonly found nucleotide(s) and another nucleotide(s) found at a specific site(s). For example, position −120 of the FCGR2B promoter sequence is polymorphic, wherein the most commonly found nucleotide at position −120 of the FCGR2B promoter is T and another nucleotide found at this polymorphic site is A. Therefore, when one of skill in the art is analyzing this site, they can determine which of the two nucleotides (T or A) is present at this site. “Polymorphism” also includes combinations of polymorphisms at more than one position in the FCGR2B promoter. Polymorphisms may provide functional differences in the genetic sequence, through changes in the encoded polypeptide, changes in mRNA stability, binding of transcriptional and translation factors to the DNA or RNA, and the like. The polymorphisms are also used as single nucleotide polymorphisms (SNPs) to detect genetic linkage to phenotypic variation in activity and expression of FCGR2B.

The present invention provides a nucleic acid comprising an FCGR2B promoter comprising SEQ ID NO:1, wherein SEQ ID NO:1 comprises one or more polymorphic sites. SEQ ID NO:1 corresponds to nucleotides −1868 to −119 of the FCGR2B promoter. SEQ ID NO: 1 also corresponds to nucleotides 1542 to 3291 of the FCGR2B nucleotide sequence provided under GenBank Accession No. AF433951. As utilized herein, “reference sequence” refers to a FCGR2B gene promoter sequence or fragment thereof comprising a specific nucleotide at a particular position(s) in the FCGR2B gene promoter sequence. Optionally, the reference is the most commonly found nucleotide or allele at the particular position or positions. This reference sequence can be a full-length FCGR2B gene promoter sequence or fragments thereof. The full length promoter sequence of FCGR2B can be found under GenBank Accession No. AF433951 and is incorporated herein in its entirety by this reference (nucleotides 1-3409 of the 12332 nucleotide sequence provided under GenBank Accession No. AF433951). References to nucleotide positions as used throughout correspond to positions of the full length FCGR2B promoter. Thus, for example, position 1 in SEQ ID NO:1 corresponds to position −1868 of the FCGR2B promoter (nucleotide 1542 of the FCGR2B nucleotide sequence provided under GenBank Accession No. AF433951), position 2 of SEQ ID NO:1 corresponds to position −1867 of the FCGR2B promoter (nucleotide 1543 of the FCGR2B nucleotide sequence provided under GenBank Accession No. AF433951), position 169 of SEQ ID NO:1 corresponds to position −1700 of the FCGR2B promoter (nucleotide 1710 of the FCGR2B nucleotide sequence provided under GenBank Accession No. AF433951), position 255 of SEQ ID NO:1 corresponds to −1614 (nucleotide 1796 of the FCGR2B nucleotide sequence provided under GenBank Accession No. AF433951), position 426 of SEQ ID NO:1 corresponds to −1443 of the FCGR2 promoter (nucleotide 1967 of the FCGR2B nucleotide sequence provided under GenBank Accession No. AF433951), position 646 of SEQ ID NO:1 corresponds to −1223 (nucleotide 2187 of the FCGR2B nucleotide sequence provided under GenBank Accession No. AF433951), position 716 of SEQ ID NO:1 corresponds to −1153 (nucleotide 2257 of the FCGR2B nucleotide sequence provided under GenBank Accession No. AF433951), position 976 of SEQ ID NO:1 corresponds to −893 (nucleotide 2517 of the FCGR2B nucleotide sequence provided under GenBank Accession No. AF433951), position 1483 of SEQ ID NO:1 corresponds to −386 (nucleotide 3024 of the FCGR2B nucleotide sequence provided under GenBank Accession No. AF433951) and position 1749 of SEQ ID NO:1 corresponds to −120 of the full length FCGR2B promoter (nucleotide 3290 of the FCGR2B nucleotide sequence provided under GenBank Accession No. AF433951).

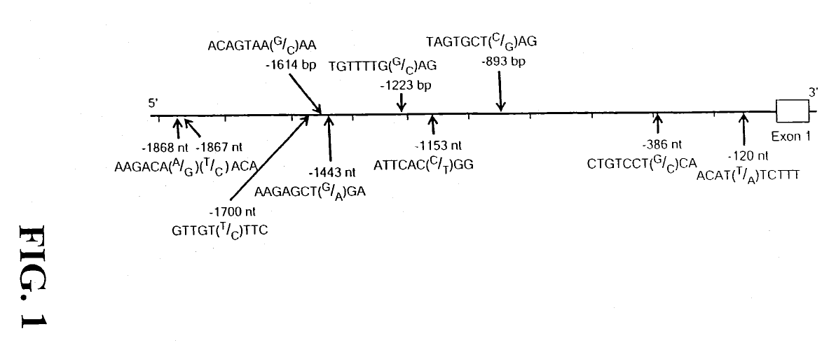

Alternatively, one of skill in the art can utilize a reference sequence or a fragment thereof comprising a nucleotide or allele that is not the most commonly found nucleotide or allele at a specific nucleotide position(s) in the FCGR2B promoter sequence or can utilize a reference sequence that comprises alternative nucleotides at a specific position(s). Therefore, one of skill in the art can utilize a FCGR2B promoter sequence that comprises such alternative nucleotides at positions −1868, −1867, −1700, −1614, −1443, −1223, −1153, −893, −386, −120 with alternative nucleotides as illustrated in FIG. 1. Therefore, when utilizing this reference sequence or a fragment thereof, the nucleotide at position −1868 can be A or G; the nucleotide at position −1867 can be T or C; the nucleotide at position −1700 can be T or C; the nucleotide at position −1443 can be G or A; the nucleotide at position −1223 can be G or C; the nucleotide at position −893 can be C or G; the nucleotide at position −386 can be G or C; and the nucleotide at position −120 can be T or A.

For example, the present invention provides a reference sequence comprising the nucleotide sequence AAGACAATACA (SEQ ID NO: 2), corresponding to nucleotides −1874 to −1864 of the FCGR2B gene promoter. This reference sequence has an “A” at position −1868, which is the most commonly found nucleotide at this position. Therefore, one of skill in the art can compare this reference sequence to a test sequence and determine if the most commonly found nucleotide (A) is present at position −1868 of the test sequence or if another nucleotide (G) is present at position −1868 of the test sequence. Alternatively, one of skill in the art could compare the test sequence to another reference sequence comprising the nucleotide sequence AAGACAA/GTACA (SEQ ID NO: 3), wherein position −1868 can be an “A” or a “G,” and determine whether the test sequence has an “A” or a “G” at position −868.

Similarly, the present invention also provides a reference sequence comprising the nucleotide sequence AAGACAATACA (SEQ ID NO: 2) or nucleotides −1874 to −1864 of the FCGR2B gene promoter. This reference sequence has a “T” at position −1867, which is the most commonly found nucleotide at this position. Therefore, one of skill in the art can compare this reference sequence to a test sequence and determine if the most commonly found nucleotide (T) is present at position −1867 or another nucleotide (C) is present at position −1867 of the test sequence. Alternatively, one of skill in the art could compare the test sequence to another reference sequence comprising the nucleotide sequence AAGACAAT/CACA (SEQ ID NO: 4), wherein position −1867 can be a “T” or a “C” and determine whether the test sequence has a “T” or a “C” at position −1867.

The present invention also provides a reference sequence comprising the nucleotide sequence GTTGTTTTC (SEQ ID NO: 5) or nucleotides −1705 to −1697 of the FCGR2B gene promoter. This reference sequence has a “T” at position −1700 which is the most commonly found nucleotide at this position. Therefore, one of skill in the art can compare this reference sequence to a test sequence and determine if the most commonly found nucleotide (T) is present at position −1700 of the test sequence or if another nucleotide (C) provided herein is present at position −1700 of the test sequence. Alternatively, one of skill in the art could compare the test sequence to another reference sequence comprising the nucleotide sequence GTTGTT/CTTC (SEQ ID NO: 6), wherein position −1700 can be a “T” or a “C,” and determine whether the test sequence has a “T” or a “C” at position −1700.

The present invention also provides a reference sequence comprising the nucleotide sequence ACAGTAAGAA (SEQ ID NO: 7) or nucleotides −1621 to −1612 of the FCGR2B gene promoter. This reference sequence has a “G” at position −1614 which is the most commonly found nucleotide at this position. Therefore, one of skill in the art can compare this reference sequence to a test sequence and determine if the most commonly found nucleotide (G) is present at position −1614 of the test sequence or if another nucleotide (C) provided herein is present at position −1614 of the test sequence. Alternatively, one of skill in the art could compare the test sequence to another reference sequence comprising the nucleotide sequence ACAGTAAG/CAA (SEQ ID NO: 8), wherein position −1614 can be a “G” or a “C,” and determine whether the test sequence has a “G” or a “C” at position −1614.

Further provided by the present invention is a reference sequence comprising the nucleotide sequence AAGAGCTGGA (SEQ ID NO: 9) or nucleotides −1450 to −1441 of the FCGR2B gene promoter. This reference sequence has a “G” at position −1443, which is the most commonly found nucleotide at this position. Therefore, one of skill in the art can compare this reference sequence to a test sequence and determine if the most commonly found nucleotide (G) is present at position −1443 of the test sequence or if another nucleotide (A) provided herein is present at position −1443 of the test sequence. Alternatively, one of skill in the art could compare the test sequence to another reference sequence comprising the nucleotide sequence AAGAGCTG/AGA (SEQ ID NO: 10), wherein position −1443 can be a “G” or an “A,” and determine whether the test sequence has a “G” or an “A” at position −1443.

Also provided by the present invention is a reference sequence comprising the nucleotide sequence TGTTTTGGAG (SEQ ID NO: 11) or nucleotides −1230 to −1221 of the FCGR2B gene promoter. This reference sequence has a “G” at position −1223, which is the most commonly found nucleotide at this position. Therefore, one of skill in the art can compare this reference sequence to a test sequence and determine if the most commonly found nucleotide (G) is present at position −1223 of the test sequence or if another nucleotide (C) provided herein is present at position −1223 of the test sequence. Alternatively, one of skill in the art could compare the test sequence to another reference sequence comprising the nucleotide sequence TGTTTTGG/CAG (SEQ ID NO: 12), wherein position −1223 can be a “G” or a “C,” and determine whether the test sequence has a “G” or an “C” at position −1223.

Further provided by the present invention is a reference sequence comprising the nucleotide sequence ATTCACCGG (SEQ ID NO: 13) or nucleotides −1159 to −1151 of the FCGR2B gene promoter. This reference sequence has a “C” at position −1153, which is the most commonly found nucleotide at this position. Therefore, one of skill in the art can compare this reference sequence to a test sequence and determine if the most commonly found nucleotide (C) is present at position −1153 of the test sequence or if another nucleotide (T) provided herein is present at position −1153 of the test sequence. Alternatively, one of skill in the art could compare the test sequence to another reference sequence comprising the nucleotide sequence ATTCACC/TGG (SEQ ID NO: 14), wherein position −1153 can be a “C” or a “T,” and determine whether the test sequence has a “C” or a “T” at position −1153.

The present invention also provides a reference sequence comprising the nucleotide sequence TAGTGCTCAG (SEQ ID NO: 15) or nucleotides −900 to −891 of the FCGR2B gene promoter. This reference sequence has a “C” at position −893, which is the most commonly found nucleotide at this position. Therefore, one of skill in the art can compare this reference sequence to a test sequence and determine if the most commonly found nucleotide (C) is present at position −893 of the test sequence or if another nucleotide (G) provided herein is present at position −893 of the test sequence. Alternatively, one of skill in the art could compare the test sequence to another reference sequence comprising the nucleotide sequence TAGTGCTC/GAG (SEQ ID NO: 16), wherein position −893 can be a “C” or a “G,” and determine whether the test sequence has a “C” or a “G” at position −893.

The present invention also provides a reference sequence comprising the nucleotide sequence CTGTCCTGCA (SEQ ID NO: 17) or nucleotides −393 to −384 of the FCGR2B gene promoter. This reference sequence has a “G” at position −386, which is the most commonly found nucleotide at this position. Therefore, one of skill in the art can compare this reference sequence to a test sequence and determine if the most commonly found nucleotide (G) is present at position −386 of the test sequence or if another nucleotide (C) provided herein is present at position −386 of the test sequence. Alternatively, one of skill in the art could compare the test sequence to another reference sequence comprising the nucleotide sequence CTGTCCTG/CCA (SEQ ID NO: 18), wherein position −386 can be a “G” or a “C,” and determine whether the test sequence has a “G” or a “C” at position −386.

Further provided by the present invention is a reference sequence comprising the nucleotide sequence ACATTTCTTT (SEQ ID NO: 19) or nucleotides −125 to −117 of the FCGR2B gene promoter. This reference sequence has a “T” at position −120, which is the most commonly found nucleotide at this position. Therefore, one of skill in the art can compare this reference sequence to a test sequence and determine if the most commonly found nucleotide (T) is present at position −129 of the test sequence or if another nucleotide (A) provided herein is present at position −120 of the test sequence. Alternatively, one of skill in the art could compare the test sequence to another reference sequence comprising the nucleotide sequence ACATT/ATCTTT (SEQ ID NO: 20), wherein position −120 can be a “T” or an “A,” and determine whether the test sequence has a “T” or an “A” at position −120.

Table 1 indicates polymorphic sites on the FCGR2B gene promoter as well as polymorphic sites in the coding regions of the FCGR2B gene, the FCGR2A gene and the FCGR3A gene. As stated above, one of skill in the art can utilize reference sequences that comprise the most commonly found allele as well as reference sequences that comprise alternative nucleotides at a specific site(s). The term “wild-type” may also be used to refer to the reference sequence comprising the most commonly found allele. It will be understood by one of skill in the art that the designation as “wild-type” is merely a convenient label for a common allele and should not be construed as conferring any particular property on that form of the sequence.

TABLE 1

| | Polymorphic Site | Polymorphic Site | Genbank | | Functional |
|---|---|---|---|---|---|
| Gene | (nt) | (aa) | Accession # | Nucleotide sequence | Consequences |
| FCGR2A | 194C→ T; 195A→ G | Gln27→ Try27 | M31932 | ACA TGC (CA/TG)G GGG GCT | Change binding |
| | | | | (SEQ ID NO: 42) | affinity |
| FCGR2A | 507G→ A | Arg131→ His131 | M31932 | TTC TCC C(G/A)T TTG GAT | Change binding |
| | | | | (SEQ ID NO: 43) | affinity |
| FCGR3A | 230T→ G→ A | Leu66→ Arg66→ His66 | X52645 | GAG AGC C(T/G/A)C ATC TCA | Change binding |
| | | | | (SEQ ID NO: 44) | affinity |
| FCGR3A | 559T→ G | Phe176→ Val176 | X52645 | GGG CTT (T/G)TT GGG AGT | Change binding |
| | | | | (SEQ ID NO: 45) | affinity |
| FCGR3A | 727A→ T | Asn232→ Tyr232 | X52645 | AAG ACA (A/T)AC ATT CGA | Unknown |
| | | | | (SEQ ID NO: 46) | |
| FCGR2B | (−1868)A→ G | NA | AF433951 | AAGACA(A/G)TACA | |
| | | | | (SEQ ID NO: 3) | |
| FCGR2B | (−1867)T→ C | NA | AF433951 | AAGACAA(T/C) ACA | |
| | | | | (SEQ ID NO: 4) | |
| FCGR2B | (−1700)T→ C | NA | AF433951 | GTTGT(T/C)TTC | |
| | | | | (SEQ ID NO: 6) | |
| FCGR2B | (−1614)G→ C | NA | AF433951 | ACAGTAA(G/C)AA | |
| | | | | (SEQ ID NO: 8) | |
| FCGR2B | (−1443)G→ A | NA | AF433951 | AAGAGCT(G/A)GA | |
| | | | | (SEQ ID NO: 10) | |
| FCGR2B | (−1223)G→ C | NA | AF433951 | TGTTTTG(G/C)AG | |
| | | | | (SEQ ID NO: 12) | |
| FCGR2B | (−1153)C→ T | NA | AF433951 | ATTCAC(C/T)GG | |
| | | | | (SEQ ID NO: 14) | |
| FCGR2B | (−893)C→ G | NA | AF433951 | TAGTGCT(C/G)AG | |
| | | | | (SEQ ID NO: 16) | |
| FCGR2B | (−386)G→ C | NA | AF433951 | CTGTCCT(G/C)CA | alters YY1 binding and |
| | | | | (SEQ ID NO: 18) | receptor expression |
| FCGR2B | (−120)T→ A | NA | AF433951 | ACAT(T/A)TCTTT | alters GATA-4 binding |
| | | | | (SEQ ID NO: 20) | and receptor expres- |
| | | | | | sion |
| FCGR2B | (775)T→ C | Ile¹⁸⁷→ Thr | AF543826 | GGGA(T/C)TGCT | change codon and |
| | | | | (SEQ ID NO: 47) | receptor function |
| FCGR2B | (208)C→ T | Thr⁻³→ Ile | AF543826 | GGGA(C/T)ACCT | change codon |
| | | | | (SEQ ID NO: 48) | |
| FCGR2B | (299)G→ A | No change | AF543826 | CGGGG(G/A)ACT | |
| | | | | (SEQ ID NO: 49) | |
| FCGR2B | (416)G→ A | No change | AF543826 | TACAC(G/A)TGC | |
| | | | | (SEQ ID NO: 50) | |
| FCGR2B | (692)G→ A | No change | AF543826 | ACGCT(G/A)TTC | |
| | | | | (SEQ ID NO: 51) | |
| FCGR2B | (846)C→ T | Pro²¹¹→ Ser | AF543826 | CTC(C/T)CAGGA | change codon |
| | | | | (SEQ ID NO: 52) | |

Nucleic acids of interest comprising the polymorphisms provided herein can be utilized as probes or primers. The complementary sequences of the nucleic acid sequences provided herein are also provided by the present invention. For the most part, the nucleic acid fragments will be of at least about 15 nt, usually at least about 20 nt, often at least about 50 nt. Such fragments are useful as primers for PCR, hybridization screening, etc. Larger nucleic acid fragments, for example, greater than about 100 nt are useful for production of promoter fragments, motifs, etc. For use in amplification reactions, such as PCR, a pair of primers will be used. The exact composition of primer sequences is not critical to the invention, but for most applications the primers will hybridize to the subject sequence under stringent conditions, as known in the art.

By “hybridizing under stringent conditions” or “hybridizing under highly stringent conditions” is meant that the hybridizing portion of the hybridizing nucleic acid, typically comprising at least 15 (e.g., 20, 25, 30, or 50 nucleotides), hybridizes to all or a portion of the provided nucleotide sequence under stringent conditions. The term “hybridization” typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize. Generally, the hybridizing portion of the hybridizing nucleic acid is at least 80%, for example, at least 90%, 95%, or 98%, identical to the sequence of or a portion of the FCGR2B promoter nucleic acid of the invention, or its complement. Hybridizing nucleic acids of the invention can be used, for example, as a cloning probe, a primer (e.g., for PCR), a diagnostic probe, or an antisense probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Assuming that a 1% mismatch results in a 1° C. decrease in the Tm, the temperature of the final wash in the hybridization reaction is reduced accordingly (for example, if sequence having >95% identity with the probe are sought, the final wash temperature is decreased by 5° C.). In practice, the change in Tm can be between 0.5° C. and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and the target nucleic acid. Additional guidance regarding such conditions is readily available in the art, for example, in Sambrook et al., 1989, Molecular Cloning, A Laboratory Manual, Cold Spring Harbor Press, NY; and Ausubel et al. (eds.), 1995, Current Protocols in Molecular Biology, (John Wiley & Sons, NY) at Unit 2.10.

The nucleic acids of the present invention can also be utilized in an array. An array may include all or a subset of the polymorphic sequences listed in FIG. 1 or in Table 1. Usually, such an array will include at least 2 different sequences. The oligonucleotide sequence on the array will usually be at least about 12 nt in length, may be the length of the provided polymorphic sequences, or may extend into the flanking regions to generate fragments of 100 to 200 nt in length. For examples of arrays, see Ramsay (1998) Nat. Biotech. 16:4044; Hacia et al. (1996) Nature Genetics 14:441-447; Lockhart et al. (1996) Nature Biotechnol. 14:1675-1680; and De Risi et al. (1996) Nature Genetics 14:457-460, which are incorporated by reference in their entirety for the methods of making and using arrays.

Nucleic acids may be naturally occurring, e.g. DNA or RNA, and may be double stranded or single stranded. Synthetic analogs of the nucleic acids are also provided. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates,where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without compromising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The present invention provides a method of characterizing a FCGR2B promoter comprising the step of identifying nucleotides at one or more polymorphic sites in the FCGR2B promoter, such identified nucleotides indicating the character of the polymorphic FCGR2B promoter. As utilized herein, the “character” of the FCGR2B promoter can be the combination of nucleotides present at polymorphic sites that make up the FCGR2B promoter haplotype as well as the biological activity associated with a particular polymorphism or combination of polymorphisms.

Some of the polymorphisms that can be identified by the methods of the present invention include, but are not limited to, polymorphisms at positions −1868, −1867, −1700, −1614, −1443, −1223, −1153, −893, −386, −120 or any combination thereof. Any individual polymorphism can be analyzed at any of these positions, or combinations of polymorphisms variants at more than one position can be identified and analyzed by the methods of the present invention.

A number of methods are available for analyzing nucleic acids for the presence of a specific sequence. For all of the methods described herein, genomic DNA can be extracted from a sample and this sample can be from any organism and can be, but is not limited to, peripheral blood, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. Such samples can be obtained directly from a subject, commercially obtained or obtained via other means. Thus, the invention described herein can be utilized to analyze a nucleic acid sample that comprises genomic DNA, amplified DNA (such as a PCR product) cDNA, cRNA, a restriction fragment or any other desired nucleic acid sample. When one performs one of the herein described methods on genomic DNA, typically the genomic DNA will be treated in a manner to reduce viscosity of the DNA and allow better contact of a primer or probe with the target region of the genomic DNA. Such reduction in viscosity can be achieved by any desired methods, which are known to the skilled artisan, such as DNase treatment or shearing of the genomic DNA, preferably lightly.

If sufficient DNA is available, genomic DNA can be used directly. Alternatively, the region of interest is cloned into a suitable vector and grown in sufficient quantity for analysis. The nucleic acid may be amplified by conventional techniques, such as the polymerase chain reaction (PCR), to provide sufficient amounts for analysis. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers flanking any of the polymorphic sites of this invention. For example, the present invention provides primers AAAGAGGGTGGAAAGGGAGGAG (SEQ ID NO: 21) or CTCTCAAAGCTTGGCGGATTCTAC (SEQ ID NO: 22), which can be utilized to amplify the region of the FCGR2B gene promoter comprising nucleotide position −386 in order to identify a polymorphism at this site. Primers TCAAGAAGCATCCAGAT (SEQ ID NO: 23) or AAACTCAGCTCAGAACCTCCTGTT (SEQ ID NO: 24) can also be utilized to amplify the region of the FCGR2B gene promoter comprising nucleotide position −120 in order to identify a polymorphism at this site. One of skill in the art would know how to design primers accordingly to amplify any region of the FCGR2B gene promoter sequence for the purposes of identifying a polymorphism at any nucleotide position throughout the FCGR2B gene promoter sequence. Amplification may also be used to determine whether a polymorphism is present by using a primer that is specific for the polymorphism.

Various methods are known in the art that utilize oligonucleotide ligation as a means of detecting polymorphisms, for examples see Riley et al (1990) Nucleic Acids Res 18:2887-2890; and Delahunty et al (1996) Am J Hum Genet 58:1239-1246, which are incorporated herein by reference in their entirety for methods of detecting polymoprhisms. Such methods include single base chain extension (SBCE), oligonucleotide ligation assay (OLA) and cleavase reaction/signal release (Invader methods, Third Wave Technologies).

LCR and Gap LCR are exponential amplification techniques, both depend on DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227, which is incorporated herein by reference in its entirety for the methods taught therein). Gap LCR (GLCR) is a version of LCR where the probes are not adjacent but are separated by 2 to 3 bases.

A method for typing single nucleotide polymorphisms in DNA, labeled Genetic Bit Analysis (GBA) has been described [Genetic Bit Analysis: a solid phase method for typing single nucleotide polymorphisms. Nikiforov T T; Rendle R B; Goelet P; Rogers Y H; Kotewicz M L; Anderson S; Trainor G L; Knapp M R. NUCLEIC ACIDS RESEARCH, (1994) 22 (20) 4167-75]. In this method, specific fragments of genomic DNA containing the polymorphic site(s) are first amplified by the polymerase chain reaction (PCR) using one regular and one phosphorothioate-modified primer. The double-stranded PCR product is rendered single-stranded by treatment with the enzyme T7 gene 6 exonuclease, and captured onto individual wells of a 96 well polystyrene plate by hybridization to an immobilized oligonucleotide primer. This primer is designed to hybridize to the single-stranded target DNA immediately adjacent from the polymorphic site of interest. Using the Klenow fragment of E. coli DNA polymerase I or the modified T7 DNA polymerase (Sequenase), the 3′ end of the capture oligonucleotide is extended by one base using a mixture of one biotin-labeled, one fluorescein-labeled, and two unlabeled dideoxynucleoside triphosphates. Antibody conjugates of alkaline phosphatase and horseradish peroxidase are then used to determine the nature of the extended base in an ELISA format. A semi-automated version of the method, which is called Genetic Bit Analysis (GBA), is being used on a large scale for the parentage verification of thoroughbred horses using a predetermined set of 26 diallelic polymorphisms in the equine genome. Additionally, minisequencing with immobilized primers has been utilized for detection of mutations in PCR products [Minisequencing: A Specific Tool for DNA Analysis and Diagnostics on Oligonucleotide Arrays. Pastinen, T. et al. Genome Research 7:606-614 (1997)].

The effect of phosphorothioate bonds on the hydrolytic activity of the 5′→3′ double-strand-specific T7 gene 6 exonuclease in order to improve upon GBA was studied [The use of phosphorothioate primers and exonuclease hydrolysis for the preparation of single-stranded PCR products and their detection by solid-phase hybridization. Nikiforov T T; Rendle R B; Kotewicz M L; Rogers Y H. PCR Methods and Applications, (1994) 3 (5) 285-91]. Double-stranded DNA substrates containing one phosphorothioate residue at the 5′ end were found to be hydrolyzed by this enzyme as efficiently as unmodified ones. The enzyme activity was, however, completely inhibited by the presence of four phosphorothioates. On the basis of these results, a method for the conversion of double-stranded PCR products into full-length, single-stranded DNA fragments was developed. In this method, one of the PCR primers contains four phosphorothioates at its 5′ end, and the opposite strand primer is unmodified. Following the amplification, the double-stranded product is treated with T7 gene 6 exonuclease. The phosphorothioated strand is protected from the action of this enzyme, whereas the opposite strand is hydrolyzed. When the phosphorothioated PCR primer is 5′ biotinylated, the single-stranded PCR product can be easily detected colorimetrically after hybridization to an oligonucleotide probe immobilized on a microtiter plate. A simple and efficient method for the immobilization of relatively short oligonucleotides to microtiter plates with a hydrophilic surface in the presence of salt can be used.

DNA analysis based on template hybridization (or hybridization plus enzymatic processing) to an array of surface-bound oligonucleotides is well suited for high density, parallel, low cost and automatable processing [Fluorescence detection applied to non-electrophoretic DNA diagnostics on oligonucleotide arrays. Ives, Jeffrey T.; Rogers, Yu Hui; Bogdanov, Valery L.; Huang, Eric Z.; Boyce-Jacino, Michael; Goelet, Philip L. L. C., Proc. SPIE-Int. Soc. Opt. Eng., 2680 (Ultrasensitive Biochemical Diagnostics), 258-269 (1996)]. Direct fluorescence detection of labeled DNA provides the benefits of linearity, large dynamic range, multianalyte detection, processing simplicity and safe handling at reasonable cost. The Molecular Tool Corporation has applied a proprietary enzymatic method of solid phase genotyping to DNA processing in 96-well plates and glass microscope slides. Detecting the fluor-labeled GBA dideoxynucleotides requires a detection limit of approx. 100 mols/μm2. Commercially available plate readers detect about 1000 mols/μm2, and an experimental setup with an argon laser and thermoelectrically-cooled CCD can detect approximately 1 order of magnitude less signal. The current limit is due to glass fluorescence. Dideoxynucleotides labeled with fluorescein, eosin, tetramethylrhodamine, Lissamine and Texas Red have been characterized, and photobleaching, quenching and indirect detection with fluorogenic substrates have been investigated.

Other amplification techniques that can be used in the context of the present invention include, but are not limited to, Q-beta amplification as described in European Patent Application No 4544610, strand displacement amplification as described in Walker et al. (1996) and EP A684 315 and, target mediated amplification as described in PCT Publication WO 9322461, the disclosures of which are incorporated herein by reference in their entirety for the methods taught therein.

Allele specific amplification can also be utilized for biallelic markers. Discrimination between the two alleles of a biallelic marker can also be achieved by allele specific amplification, a selective strategy, whereby one of the alleles is amplified without amplification of the other allele. For allele specific amplification, at least one member of the pair of primers is sufficiently complementary with a region of a FCGR2B gene promoter sequence comprising the polymorphic base of a biallelic marker of the present invention to hybridize therewith. Such primers are able to discriminate between the two alleles of a biallelic marker. This can be accomplished by placing the polymorphic base at the 3′ end of one of the amplification primers. Such allele specific primers tend to selectively prime an amplification or sequencing reaction so long as they are used with a nucleic acid sample that contains one of the two alleles present at a biallelic marker because the extension forms from the 3′ end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well with the ordinary skill in the art.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, can be analyzed by one of a number of methods known in the art. The nucleic acid can be sequenced by dideoxy or other methods. Hybridization with the variant sequence can also be used to determine its presence, by Southern blots, dot blots, etc. The hybridization pattern of a control (reference) and variant sequence to an array of oligonucleotide probes immobilized on a solid support, as described in U.S. Pat. No. 5,445,934 and WO95/35505, which are incorporated herein by reference in their entirety for the methods, may also be used as a means of detecting the presence of variant sequences. Single strand conformational polymorphism (SSCP) analysis, denaturing gradient gel electrophoresis (DGGE), mismatch cleavage detection, and heteroduplex analysis in gel matrices are used to detect conformational changes created by DNA sequence variation as alterations in electrophoretic mobility. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels.

The present invention also provides an array of oligonucleotides for identification of polymorphisms, where discrete positions on the array are complementary to one or more of the provided polymorphic sequences, e.g. oligonucleotides of at least 12 nt, frequently 20 nt, or larger, and including the sequence flanking the polymorphic position. Such an array may comprise a series of oligonucleotides, each of which can specifically hybridize to a different polymorphism of the present invention. As mentioned above, an array may include all or a subset of the sequences listed in FIG. 1 or Table 1. Usually such an array will include at least 2 different polymorphic sequences, i.e. polymorphisms located at unique positions within the locus, and may include all of the provided polymorphisms. Therefore, the array can include sequences comprising the most commonly found allele at a position as well as other nucleotides found at this position. The array can optionally comprise the most commonly found allele at a second, third, fourth, fifth, or more positions as well as other nucleotides at each of these positions. Each oligonucleotide sequence on the array will usually be at least about 12 nt in length (i.e., 10-15 nt), may be the length of the provided polymorphic sequences, or may extend into the flanking regions to generate fragments of 100 to 200 nt in length.

The present invention also provides the use of the nucleic acid sequences of the invention in methods using a mobile solid support to analyze polymorphisms. See for example, WO 01/48244 which is incorporated herein by reference in its entirety for the methods taught therein. The method of performing a Luminex FlowMetrix-based SNP analysis involves differential hybridization of a PCR product to two differently-colored FACS-analyzable beads. The FlowMetrix system currently consists of uniforrnly-sized 5 micron polystyrene-divinylbenzene beads stained in eight concentrations of two dyes (orange and red). The matrix of the two dyes in eight concentrations allows for 64 differently-colored beads (82) that can each be differentiated by a FACScalibur suitably modified with the Luminex PC computer board. In the Luminex SNP analysis, covalently-linked to a bead is a short (approximately 18-20 bases) “target” oligodeoxynucleotide (oligo). The nucleotide positioned at the center of the target oligo encodes the polymorphic base. A pair of beads are synthesized; each bead of the pair has attached to it one of the polymorphic oligonucleotides. A PCR of the region of DNA surrounding the to-be analyzed SNP is performed to generate a PCR product. Conditions are established that allow hybridization of the PCR product preferentially to the bead on which is encoded the precise complement. In one format (“without competitor”), the PCR product itself incorporates a flourescein dye and it is the gain of the flourescein stain on the bead, as measured during the FACScalibur run, that indicates hybridization. In a second format (“with competitor,”) the beads are hybridized with a competitor to the PCR product. The competitor itself in this case is labeled with flourescein. And it is the loss of the flourescein by displacement by unlabeled PCR product that indicates successful hybridization.

The present invention also provides a method for determining a FCGR2B promoter haplotype in a human subject comprising identifying a nucleotide present at one or more polymorphic sites in in either or both copies of the FCGR2B promoter contained in the subject genomic nucleic acid, wherein the nucleotide present at the polymorphic site or sites indicates the FCGR2B promoter haplotype. It will be recognized by one of skill in the art that numerous haplotypes are possible. Therefore, one of skill in the art can determine the impact of each haplotype on FCGR2B levels and FCGR2B activity as described in the Examples.

For example, one of skill in the art could identify the nucleotide present in either or both copies of the FCGR2B promoter contained in the subject genomic nucleic acid at position −386 or at position −120, and determine a subject's FCGR2B promoter haplotype. The haplotypes for this particular analysis can be −386C/−120A, −386G/−120T, −386G/−120A. −386C/−120T. Similarly, one of skill in the art could identify the nucleotide in a FCGR2B promoter nucleic acid sequence at position −1868, −1867, −1700, −1614, −1443, −1223, −1153 or −893 and determine the FCGR2B promoter haplotype. Therefore, any of positions −1868, −1867, −1700, −1614, −1443, −1223, −1153, −893, −396 or −120 can be analyzed individually or in combination to obtain the haplotypes of the present invention.

The present invention also provides a method for determining a FCGR2B promoter haplotype in a population of human subjects comprising identifying a nucleotide present at a one or more polymorphic sites in either or both copies of the promoter contained in the subjects' genome, wherein the nucleotide present at the polymorphic site or sites indicates the promoter haplotype of each subject.

Each haplotype can be correlated with FCGR2B levels to generate a database of reference haplotypes, such that one of skill in the art can compare a subject's haplotype to a reference haplotype or haplotypes and determine, for example, whether the subject is at risk for developing an inflammatory disease, such as an autoimmune disorder. As set forth below, one of skill in the art can also establish correlations between FCGR2B haplotypes and other physiological and/or clinical manifestations of variable FcγRIIB function or expression. These include incidence of disease caused by infections (e.g., viral, bacterial, fungal), presence of cancer, and vaccine efficacy. The correlation can further utilize haplotypes of related genes like FCGR2A or polymorphisms in spectifc regions of FCGR2B or FCGR2A.

As used herein, “autoimmune disorder” describes a disease state or syndrome whereby a subject's body produces a dysfunctional immune response against the subject's own body components, with adverse effects. This may include production of B cells which produce antibodies with specificity for all antigens, allergens or major histocompatibility (MHC) antigens, or it may include production of T cells bearing receptors that recognize self-components and produce cytokines that cause inflammation. Examples of autoimmune diseases include, but are not limited to, ulcerative colitis, Crohn's disease, multiple sclerosis, rheumatoid arthritis, diabetes mellitus, pernicious anemia, autoimmune gastritis, psoriasis, Bechet's disease, Wegener's granulomatosis, Sarcoidois, autoimmune thyroiditis, autoimmune oophoritis, bullous pemphigoid, phemphigus, polyendocrinopathies, Still's disease, Lambert-Eaton myasthenia syndrome, myasthenia gravis, Goodpasture's syndrome, autoimmune orchitis, autoimmune uveitis, systemic lupus erythematosus, Sjogren's Syndrome and ankylosing spondylitis.

Therefore, the present invention provides a method of determining a subject's predisposition to an inflammatory disease comprising comparing the subject's FCGR2B promoter haplotype with one or more reference promoter haplotypes that correlate with elevated FcγRIIb levels, a similar haplotype in the subject's FCGR2B promoter as compared to the reference promoter haplotype or haplotypes indicating a predisposition to the inflammatory disease. By “predisposition to an inflammatory disease” is meant an increased likelihood of developing the disease in the future as compared to the general population or a reference subset thereof.

The methods of the present invention are suitable in diagnosis, staging, prognostication and treatment of an inflammatory disease. Any statistically significant correlation that is found to exist between FCGR2B promoter haplotypes (or combinations of FCGR2B promoter haplotypes, FCGR2B haplotypes, and FCGR3A haplotypes) and a clinical parameter can be used to determine susceptibility to an inflammatory disease, recurrence of an inflammatory disease, responsiveness to anti-inflammatory treatment and duration of an anti-inflammatory disease.

The present method of determining a subject's predisposition to an inflammatory disease comprising comparing the subject's FCGR2B promoter haplotype with one or more reference promoter haplotypes that correlate with elevated FcγRIIb levels can be combined with an analysis of additional genetic correlates of such predisposition. For example, the method can further comprise comparing the subject's FCGR3A extracellular domain with one or more reference polymorphic extracellular domain sequences that correlate with reduced FcγRIIIa activity, a similar extracellular domain in the subject's FCGR3A extracellular domain as compared to the reference extracellular domain sequences further indicating a predisposition to the inflammatory disease. For example, one of skill in the art can compare a subject's FCGR2B haplotype to reference FCGR2B haplotypes and compare a subject's FCGR3A extracellular domain coding sequence to an FCGR3A extracellular domain comprising a polymorphism at position 559 and determine if there is a T or a G at position 559. It there is a G at position 559, this means that the subject has a polymorphic version of FCGR3A and the phenylalanine most commonly found at position 176 is a valine in the subject. This FCGR3A polymorphism correlates with a predisposition to an inflammatory disease. Therefore, in combination with a FCGR2B haplotype that correlates with a predisposition to an inflammatory disease, this FCGR3A polymorphism would provide further indication that a subject is predisposed to an inflammatory disease. The present method of determining a subject's predisposition to an inflammatory disease comprising comparing the subject's FCGR2B promoter haplotype with one or more reference promoter haplotypes that correlate with elevated FcγRIIb activity can further comprise comparing the subject's FCGR2B transmembrane domain with one or more reference polymorphic transmembrane domains that correlate with increased FcγRIIb levels, a similar transmembrane domain as compared to the reference polymorphic transmembrane domains further indicating a predisposition to the inflammatory disease.

For example, one of skill in the art can compare a subject's FCGR2B haplotype to reference FCGR2B haplotypes and to an FCGR2B transmembrane domain comprising a polymorphism at position 775 and determine if there is a T or a C at position 775. It there is a C at position 775, this means that the isoleucine most commonly found at position 187 is a threonine in the subject. This FCGR2B polymorphism correlates, for example, with a predisposition to an inflammatory disease. Therefore, in combination with a FCGR2B promoter haplotype that correlates with a predisposition to an inflammatory disease, this FCGR2B polymorphism would provide further indication that a subject is predisposed to an inflammatory disease.

The present method of determining a subject's predisposition to an inflammatory disease comprising comparing the subject's FCGR2B promoter haplotype with one or more reference promoter haplotypes that correlate with elevated FcγRIIb levels can further comprise comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating a predisposition to the inflammatory disease.

The present invention also provides a method of determining a subject's predisposition to an inflammatory disease comprising comparing the subject's FCGR2B promoter haplotype with one or more reference promoter haplotypes that correlate with elevated FcγRIIb levels can further comprise one, two or three of the following: a) comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating a predisposition to the inflammatory disease; b) comparing the subject's FCGR2B transmembrane domain with one or more reference polymorphic transmembrane domains that correlate with increased FcγRIIb activity, a similar transmembrane domain as compared to the reference transmembrane domains further indicating a predisposition to the inflammatory disease; and c) comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating a predisposition to the inflammatory disease.

The present invention also provides a method of determining a subject's susceptibility to an infection comprising comparing the subject's FCGR2B promoter haplotype with one or more reference promoter haplotypes that correlate with elevated FcγRIIb levels, a similar haplotype in the subject's FCGR2B promoter as compared to the reference promoter haplotype or haplotypes indicating the subject's susceptibility to an infection. By “susceptibility to an infection” is meant an increased likelihood of developing symptoms of the infection as compared to the general population or a reference subset thereof.

The methods of the present invention are suitable for diagnosis, staging, prognostication and treatment of infections (e.g., viral, bacterial, and fungal). Any statistically significant correlation that is found to exist between FCGR2B promoter haplotypes (or combinations of FCGR2B promoter haplotypes and FCGR3A haplotypes) and a clinical parameter can be used to determine susceptibility to infection, recurrence of infection, responsiveness to antibiotics or antiviral, antibacterial, or anti-fungal agents and duration of infection. Bacterial infection include, but are not limited to, Streptococcus, Staphylococcus, Pneumococcus and Hemophilus influenzae. Viral infections include, but are not limited to, those caused by influenza virus, adenoviruses, human immunodeficiency virus.

As described above for methods of determining a subject's predisposition to an autoimmune disease, the method of determining a subject's susceptibility to an infection comprising comparing the subject's FCGR2B promoter haplotype with one or more reference promoter haplotypes that correlate with elevated FcγRIIb levels can further comprising one or more of the following steps: (a) comparing the subject's FCGR3A extracellular domain with one or more reference polymorphic extracellular domain sequences that correlate with reduced FcγRIIIa activity, a similar extracellular domain in the subject's FCGR3A extracellular domain as compared to the reference extracellular domain sequences further indicating the subject's susceptibility to an infection; (b) comparing the subject's FCGR2B transmembrane domain with one or more reference polymorphic transmembrane domains that correlate with increased FcγRIIb activity, a similar transmembrane domain as compared to the reference polymorphic transmembrane domains further indicating the subject's susceptibility to an infection; (c) comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating the subject's susceptibility to an infection.

The present invention also provides a method of determining a subject's ability to mount an immune response comprising comparing the subject's FCGR2B promoter haplotype with one or more reference promoter haplotypes that correlate with elevated FcγRIIb levels, a similar haplotype in the subject's FCGR2B promoter as compared to the reference promoter haplotype or haplotypes indicating the subject's ability to mount an immune response. By “ability to mount an immune response” is meant an increased likelihood of activating lymphocytes, developing antibodies, and displaying other parameters of an immune response as compared to the general population or a reference subset thereof. An “inability” or “reduced ability to mount an immune response,” as used herein, refers to a reduced likelihood of activating lymphocytes, developing antibodies, and displaying other parameters of an immune response as compared to the general population or a reference subset thereof. Thus, when a subject's ability is said to correlate with an elevated FcγRIIb level, for example, or an increase in a similar correlate, it should be clear that a reduced FcγRIIb level, for example, or a reduction in a similar other correlate would indicate an inability to mount an immune response.

The method of determining a subject's ability to mount an immune response comprising comparing the subject's FCGR2B promoter haplotype with one or more reference promoter haplotypes that correlate with elevated FcγRIIb levels can further comprise one or more of the following: (a) comparing the subject's FCGR3A extracellular domain with one or more reference polymorphic extracellular domain sequences that correlate with reduced FcγRIIIa activity, a similar extracellular domain in the subject's FCGR3A extracellular domain as compared to the reference extracellular domain sequences further indicating the subject's ability to mount an immune response; (b) comparing the subject's FCGR2B transmembrane domain with one or more reference polymorphic transmembrane domains that correlate with increased FcγRIIb activity, a similar transmembrane domain as compared to the reference polymorphic transmembrane domains further indicating the subject's ability to mount an immune response; or (c) comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating the subject's subject's ability to mount an immune response.

Since subjects will vary depending on numerous parameters including, but not limited to, race, age, weight, medical history etc., as more information is gathered on populations, the database can contain haplotype information classified by race, age, weight, medical history etc., such that one of skill in the art can assess the subject's risk of developing an inflammatory disease, the subject's susceptibility to an infection, the subject's ability to mount an immune response and/or the subject's responsiveness to a therapeutic agent based on information more closely associated with the subject's demographic profile. Where there is a differential distribution of a polymorphism by racial background or another parameter, guidelines for drug administration can be generally tailored to a particular group.

The present invention provides a computer system comprising a) a database including records comprising a plurality of reference haplotypes comprising the SNPs of Table 1 and associated diagnosis and therapy data; and b) a user interface capable of receiving a selection of one or more test haplotypes for use in determining matches between the test haplotypes and the reference haplotypes and displaying the records associated with matching haplotypes.

It will be appreciated by those skilled in the art that the nucleic acids provided herein as well as the nucleic acid sequences identified from subjects can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate a list of sequences comprising one or more of the nucleic acids of the invention. Another aspect of the present invention is a computer readable medium having recorded thereon at least 2, 5, 10, 15, 20, 25, 30, 50, 100, 200, 250, 300, 400, 500, 1000, 2000, 3000, 4000 or 5000 nucleic acids of the invention or nucleic acid sequences identified from subjects.

Computer readable media include magnetically readable media, optically readable media, electronically readable media and magnetic/optical media. For example, the computer readable media may be a hard disc, a floppy disc, a magnetic tape, CD-ROM, DVD, RAM, or ROM as well as other types of other media known to those skilled in the art.

Embodiments of the present invention include systems, particularly computer systems which contain the sequence information described herein. As used herein, “a computer system” refers to the hardware components, software components, and data storage components used to store and/or analyze the nucleotide sequences of the present invention or other sequences. The computer system preferably includes the computer readable media described above, and a processor for accessing and manipulating the sequence data.

Preferably, the computer is a general purpose system that comprises a central processing unit (CPU), one or more data storage components for storing data, and one or more data retrieving devices for retrieving the data stored on the data storage components. A skilled artisan can readily appreciate that any one of the currently available computer systems are suitable.

In one particular embodiment, the computer system includes a processor connected to a bus which is connected to a main memory, preferably implemented as RAM, and one or more data storage devices, such as a hard drive and/or other computer readable media having data recorded thereon. In some embodiments, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In some embodiments, the data storage component is a removable computer readable mediun such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device. Software for accessing and processing the nucleotide sequences of the nucleic acids of the invention (such as search tools, compare tools, modeling tools, etc.) may reside in main memory during execution.

In some embodiments, the computer system may further comprise a sequence comparer for comparing the nucleic acid sequences stored on a computer readable medium to another test sequence stored on a computer readable medium. A “sequence comparer” refers to one or more programs which are implemented on the computer system to compare a nucleotide sequence with other nucleotide sequences.

Accordingly, one aspect of the present invention is a computer system comprising a processor, a data storage device having stored thereon a nucleic acid of the invention, a data storage device having retrievably stored thereon reference nucleotide sequences to be compared with test or sample sequences and a sequence comparer for conducting the comparison. The sequence comparer may indicate a homology level between the sequences compared or identify a difference between the two sequences. For example, a reference sequence comprising SEQ ID NO: 1 or any fragment thereof can be compared with a test sequence from a subject to determine if the test sequence is the same as or different the reference sequence.

Alternatively, the computer program may be a computer program which compares a test nucleotide sequence(s) from a subject or a plurality of subjects to a reference nucleotide sequence(s) in order to determine whether the test nucleotide sequence(s) differs from or is the same as a reference nucleic acid sequence(s) at one or more nucleotide positions. Optionally such a program records the length and identity of inserted, deleted or substituted nucleotides with respect to the sequence of either the reference polynucleotide or the test nucleotide sequence. In one embodiment, the computer program may be a program which determines whether the nucleotide sequences of the test nucleotide sequence contains one or more single nucleotide polymorphisms (SNP) with respect to a reference nucleotide sequence. These single nucleotide polymorphisms may each comprise a single base substitution, insertion, or deletion.

Accordingly, another aspect of the present invention is a method for determining whether a test nucleotide sequence differs at one or more nucleotides from a reference nucleotide sequence comprising the steps of reading the test nucleotide sequence and the reference nucleotide sequence through use of a computer program which identifies differences between nucleic acid sequences and identifying differences between the test nucleotide sequence and the reference nucleotide sequence with the computer program.

The computer program can be a program which identifies single nucleotide polymorphisms. The method may be implemented by the computer systems described above. The method may also be performed by reading at least 2, 5, 10, 15, 20, 25, 30, 50, 100, or more test nucleotide sequences and the reference nucleotide sequences through the use of the computer program and identifying differences between the test nucleotide sequences and the reference nucleotide sequences with the computer program. A computer program that identifies single nucleotide polymorphisms in a FGR2B gene promoter sequence and determines a subject's haplotype is also contemplated by this invention. This invention also provides for a computer program that correlates haplotypes with FCGR2B levels such that one of skill in the art can assess a subject's risk of developing an inflammatory disease, susceptibility to infection, a subject's ability to mount an immune response and/or a subject's responsiveness to a therapeutic agent, such as an immunoglobulin based therapy. The computer program can optionally include treatment options or drug indications for subjects with haplotypes associated with increased risk of inflammatory disease, increased susceptibility to infection, decreased or increased ability to mount an immune response and/or increased or decreased responsiveness to a therapeutic agent.

The computer program could similarly compare amino acid sequences encoded by the relevant nucleic acid sequences.

The present invention provides a method of determining a subject's responsiveness to a therapeutic agent (e.g., an immunoglobulin based therapeutic agent) comprising comparing the subject's FCGR2B promoter haplotype with one or more reference promoter haplotypes that correlate with modulated FcγRIIb levels, a similar haplotype in the subject's FCGR2B promoter as compared to the reference promoter haplotype or haplotypes indicating the subject's responsiveness to the therapeutic agent.

By “modulate” as used herein is meant to increase or decrease as compared to a control level. The control level is generally determined in this context to an average population or a subset thereof. By “responsiveness” is meant an ability to respond. Generally, responsiveness refers to an ability to respond like or beter than the control response. As used herein, correlates of responsivness will not correlate with reduced responsiveness or unresponsiveness. Rather, if an increase in FcγRIIb levels correlates with responsiveness than a decrease in FcγRIIb levels would correlate with reduced responsiveness.

The immunoglobulin based therapeutic agents of the present invention include, but are not limited to, monoclonal antibodies (such as Rituximab), Fc fusion proteins and intravenous gammaglobulin.

For example, if the subject's FCGR2B promoter haplotype is similar to one or more reference promoter haplotypes that correlate with decreased FcγRIIb levels, this would indicate that the subject is more responsive to an immunoglobulin based therapeutic agent that acts via antibody-dependent cellular cytotoxicity (ADCC). However, if the subject's FCGR2B promoter haplotype is similar to one or more reference promoter haplotypes that correlate with increased FcγRIIb levels, this would indicate that the subject is more responsive to an immunoglobulin based therapeutic agent that acts via cross-linking and activation of an endogenous cell program, in the target cell, such as apoptosis. Therefore, one of skill in the art would be able to select an appropriate therapeutic agent, adjust the dose of the therapeutic agent and predict the clinical response based on the FCGR2B promoter haplotype of the subject.

The present method of determining a subject's responsiveness to an immunoglobulin based therapeutic agent comprising comparing the subject's FCGR2B promoter haplotype with one or more reference promoter haplotypes that correlate with modulated FcγRIIb levels can further comprise comparing the subject's FCGR3A extracellular domain coding sequence with one or more reference extracellular domain polymorphic sequences that correlate with modulated FcγRIIIa activity, a similar extracellular domain in the subject's FCGR3A extracellular domain as compared to the reference extracellular domain sequences further indicating the subject's responsiveness to an immunoglobulin based therapeutic agent. Thus, a reduction in FcγRIIIa binding avidity for immunoglobulin would correlate with a subject's responsiveness to certain immunoglobulin based therapies, including for example Rituximab. Responsiveness to antibodies that work through ADCC, however, would correlate with an increase in FcγRIIIa avidity for binding.

Similarly, the method of determining a subject's responsiveness to a therapeutic agent (like an immunoglobulin based therapeutic agent) comprising comparing the subject's FCGR2B promoter haplotype with one or more reference promoter haplotypes that correlate with modulated FcγRIIb levels can further comprise (a) comparing the subject's FCGR2B transmembrane domain with one or more reference polymorphic transmembrane domains that correlate with modulate FcγRIIb activity, a similar transmembrane domain as compared to the reference transmembrane domains further indicating the subject's responsiveness to an immunoglobulin based therapeutic agent or (b) comparing the subject's FCGR3A cytoplasmic domain with one or more reference polymorphic FCGR3A cytoplasmic domains that correlate with modulated FcγRIIIa activity, a similar cytoplasmic domain as compared to the reference cytoplasmic domains further indicating the subject's responsiveness to an immunoglobulin based therapeutic agent. One of skill in the art can also compare the subjects FCGR2A coding sequence with one or more reference polymorphic FCGR2A sequence (for example, the polymorphic sequences set forth in Table 1) that correlate with modulated FcγRIIa activity, a similar coding sequence as compared to the reference sequence further indicating the subject's responsiveness to an immunoglobulin based therapeutic.

The present invention also provides a method of selecting a population of human subjects for a treatment with a immunoglobulin based therapeutic agent comprising the steps of a) determining each potential subject's responsiveness to the immunoglobulin based therapeutic agent according to any of the methods disclosed herein; and b) selecting those subjects with responsiveness to the immunoglobulin based therapeutic agent. Such methods would be useful in selecting therapy for a particular subject and for selecting a population of subjects for a clinical trial.

Also provided by the present invention is a method of selecting a therapy or treatment for a disorder in a subject, comprising the steps of determining a FCGR2B promoter haplotype in the subject according to the method described herein and selecting the treatment based on the FCGR2B promoter haplotype. Such analysis can be combined with the analysis of FCGR2B and FCGR3A haplotypes. By “selecting therapy or treatment” is meant the type of treatment, route or frequency of administration, or dosage. Thus, a subject identified to have a reduced responsiveness to a particular treament could be treated with a higher dose, more frequent administrations, or a different treatment entirely.

Further provided is a method of selecting a therapy or treatment for a subject, the method comprising: a) comparing the FCGR2B promoter haplotype of the subject to a plurality of reference FCGR2B promoter haplotypes, wherein each reference FCGR2B promoter haplotype has a value, each value corresponding to a selected therapy; and b) selecting the reference FCGR2B promoter haplotype most similar to the subject's FCGR2B promoter haplotype, to thereby select a therapy for the subject. Such analysis can be combined with the analysis of FCGR2B and FCGR3A haplotypes.

The FCGR2B promoter haplotype of the subject can be utilized in combination with the SNPs, if present, in the subject's FCGR3A receptor and/or with the SNPs, if present, in the coding region for the transmembrane domain of the subject's FCGR2B receptor to select a therapy and/or dosage for the subject.

The disorder can be any disorder found to correlate with a FCGR2B promoter haplotype such as an inflammatory disease, cancer or infection. The treatment or therapy can be, but is not limited to, an anti-inflammatory agent, an anti-cancer agent, an antiviral agent, an antibacterial agent, a vaccine or an immunoglobulin-based therapeutic. Combinations of these agents can also be used to treat a subject. The treatment can also be an agent that modulates FcγRIIIa levels or activity. For example, one of skill in the art can administer an agent that increases FcγRIIIa levels in order to increase responsiveness to a therapeutic agent, such as an immunoglobulin based therapy, in a subject. This agent can be used in combination with any other therapeutic agent described herein.

The present invention provides a method of identifying a compound that modulates FcγRIIb levels comprising: a) contacting with a test compound a cell containing a FCGR2B promoter nucleic acid sequence comprising selected nucleotides at one or more polymorphic sites at residues −386 and −120 in the FCGR2B promoter, wherein the promoter nucleic acid sequence is operatively linked to a nucleic acid sequence encoding a reporter protein; b) detecting the amount of reporter protein expressed by the cell after contact with the test compound; and c) comparing the amount of reporter protein in the contacted cell with the amount of reporter protein in a control cell, an increased or decreased amount of reporter protein in the test cell as compared to the control cell indicating a compound that modulates FcγRIIb levels. The contacting step can occur in vivo (e.g., in a test animal) or in vitro.

Optionally, the control cell is not contacted by the test compound or the control cell is the treated cell before or after the contacting step when the treatment has no remaining effect on the cell.

Also provided by this invention is a method of making a pharmaceutical composition that modulates FcγRIIb levels comprising: a) contacting with a test compound a cell containing a FCGR2B promoter nucleic acid sequence comprising selected nucleotides at one or more polymorphic sites at residues −386 and −120 in the FCGR2B promoter, wherein the promoter nucleic acid sequence is operatively linked to a nucleic acid sequence encoding a reporter protein; b) detecting the amount of reporter protein expressed by the cell after contact with the test compound; and c) comparing the amount of reporter protein in the contacted cell with the amount of reporter protein in a control cell, an increased or decreased amount of reporter protein in the test cell as compared to the control cell indicating a compound that modulates FcγRIIb levels; and d) placing the compound in a pharmaceutically acceptable carrier.

By “pharmaceutically acceptable carrier” is meant a material that is not biologically or otherwise undesirable, i.e., the material may be administered to a subject, along with a nucleic acid or along with a modulator of FcγRIIb identified or made by the methods taught herein, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The present invention thus relates to a method of preventing or reducing the effects of inflammatory diseases, infection, cancer etc. with a composition that modulates FcγRIIb levels.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the composition. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated, the particular nucleic acid or modulator used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release. Compositions including the liposomes in a pharmaceutically acceptable carrier are also contemplated.

Transdermal delivery devices have been employed for delivery of low molecular weight proteins by using lipid-based compositions (i.e., in the form of a patch) in combination with sonophoresis. However, as reported in U.S. Pat. No. 6,041,253 to Ellinwood, Jr. et al., which is hereby incorporated by reference in its entirety, transdermal delivery can be further enhanced by the application of an electric field, for example, by ionophoresis or electroporation. Using low frequency ultrasound which induces cavitation of the lipid layers of the stratum comeum, higher transdermal fluxes, rapid control of transdermal fluxes, and drug delivery at lower ultrasound intensities can be achieved. Still further enhancement can be obtained using a combination of chemical enhancers and/or magnetic field along with the electric field and ultrasound.

Implantable or injectable protein depot compositions can also be employed, providing long-term delivery of the composition. For example, U.S. Pat. No. 6,331,311 to Brodbeck, which is hereby incorporated by reference in its entirety, reports an injectable depot gel composition which includes a biocompatible polymer, a solvent that dissolves the polymer and forms a viscous gel, and an emulsifying agent in the form of a dispersed droplet phase in the viscous gel. Upon injection, such a gel composition can provide a relatively continuous rate of dispersion of the agent to be delivered, thereby avoiding an initial burst of the agent to be delivered.

The test compound and modulator taught herein can be, but is not limited to, antibodies, chemicals, small molecules, antisense RNAs, siRNAs, drugs and secreted proteins. Test compounds in the form of cDNAs which express in the cells of these methods can also be tested in the methods of the present invention.

As used herein, a “reporter protein” is any protein that can be specifically detected when expressed. Reporter proteins are useful for detecting or quantifying expression from expression sequences. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), green reef coral fluorescent protein (G-RCFP), cyan fluorescent protein (CFP), red fluorescent protein (RFP) and yellow fluorescent protein (YFP).

The following examples are put forth so as to provide those of ordinary skill in the art with a complete disclosure and description of how the antibodies, polypeptides, nucleic acids, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

### EXAMPLE I

### Regulatory FCGR2B Polymorphisms and their Association With Systemic Lupus Erythematosus

FcγRIIb, the ITIM-containing receptor for immunoglobulin G, (MIM 604590) plays an important role in maintaining the homeostasis of immune responses. The present invention provides the identification of 10 novel single nucleotide polymorphisms (SNPs) in the promoter region of human FCGR2B gene and the characterization of two functionally distinct haplotypes in its proximal promoter. In luciferase reporter assays, the less frequent promoter haplotype leads to increased expression of the reporter gene in both B lymphoid and myeloid cell lines under constitutive and stimulated conditions. Four independent genome wide scans support linkage of the human Fcγ receptor region to the systemic lupus erythematosus (SLE, OMIM 152700) phenotype. A case-control study in 600 Caucasians indicates a significant association of the less frequent FCGR2B promoter haplotype with the SLE phenotype (odds ratio=1.65, P=0.0054). The FCGR2B haplotype has no linkage disequilibrium with previously identified FCGR2A and FCGR3A polymorphisms and after adjustment for FCGR2A and FCGR3A, FCGR2B showed a persistent association with SLE (odds ratio=1.72, P=0.0083). These results show that an expression variant of FCGR2B can be a risk factor for human lupus and implicate FCGR2B in disease pathogenesis.

Within the classical IgG Fc-binding receptor family, FcγRIIb (CD32B) is the only receptor that bears an immuno-receptor tyrosine-based inhibitory motif (ITIM) in its cytoplasmic domain (1). FcγRIIb is expressed on B lymphocytes, myeloid cell lineages, dendritic and mast cells. On B lymphocytes, co-ligation of FcγRIIb with the B cell antigen receptor (BCR) by IgG immune complexes downregulates BCR signaling and modulates the threshold for B cell activation and proliferation (2-6). Co-ligation of FcγRIIb also provides a negative feedback mechanism for immunoglobulin (Ig) production by B cells. On myeloid lineage cells, FcγRIIb co-clustering with the activating Fcγ receptors, such as FcγRIa (CD64), FcγRIIa (CD32A), and FcγRIIIa (CD16A), down-modulates their function (2). Antibody-mediated phagocytosis by macrophages is decreased by exaggerated FcγRIIb co-clustering and is enhanced by disruption of FcγRIIb (7-9). On follicular dendritic cells (FDC), FcγRIIb mediates the retention and conversion of immune complexes to a highly immunogenic form, which facilitate B cell recall responses (10-13). Thus, FcγRIIb plays multiple roles in modulating immune function and thus maintaining immune homeostasis. Indeed, studies in mouse models have highlighted the role of FCGR2B in the development of autoimmune diseases (14-19). For example, targeted disruption of FCGR2B in the mouse leads to elevated serum Ig levels and, on the susceptible C57BL/6 background, leads to the development of lupus-like phenotypes (20, 21).

Human SLE is a prototypic autoimmune disease characterized by production of antinuclear autoantibodies and tissue deposition of immune complexes (22-25). This complex polygenic disease has strong genetic components (λs≈20) (26, 27). In humans, outside of MHC class II, genetic polymorphisms or defects in genes involved in antigen uptake, processing and immune complex clearance such as complement, FCGR2A and FCGR3A have been identified to contribute to SLE susceptibility (26, 28-33). Recently, programmed cell death gene 1 (PDCD1) which regulates B cell activation has been identified as an autoimmunity candidate gene in the mouse (34, 35), and a single nucleotide polymorphism in a putative RUNX1 binding site in the promoter of human PDCD1 gene has been implicated as a risk allele for SLE (34, 35). However, potential variations in the regulatory regions of human FCGR2B as a disease susceptibility gene have not yet been characterized.

Unlike the single nucleotide polymorphisms (SNPs) in human FCGR2A and FCGR3A which affect the ligand-binding properties of the receptors (29, 36), no non-synonymous SNPs encoding the extracellular domains of FcγRIIb in more than 120 donors were found in the studies presented herein. However, 10 polymorphic sites were identified in the 2 kb promoter region of human FCGR2B which defined two SNP haplotypes in its proximal promoter. In luciferase reporter assays, the less frequent variant FCGR2B haplotype increases the promoter activity both constitutively and under inducible conditions. In a case-control study of 600 Caucasians, the variant FCGR2B haplotype is significantly associated with the SLE phenotype. This association is not due to the effects of previously identified FCGR2A or FCGR3A polymorphisms. This observation not only provides evidence for the genetic association of FCGR2B with human lupus but also is the first study to characterize the functionally important promoter polymorphisms in FCGR2B, one of the key regulators in immune responses.

### Donors

Caucasian SLE patients and controls were recruited as part of the University of Alabama at Birmingham-based DISCOVERY cohort and as part of the Carolina Lupus Study (37), a population-based case-control study. The studies were reviewed and approved by the Institution Review Board, and all donors provided written informed consent.

### FCGR2B Genotyping

Long-range polymerase chain reaction (PCR) was performed to specifically amplify FCGR2B from genomic DNA using Failsafe PCR system (Epicenter Technologies, Madison, Wis.). The sense primer (5′-CTCCACAGGTTACTCGTTTCTACCTTA TCTTAC-3′) (SEQ ID NO: 25) anneals at both FCGR2B/C −2 kb promoters, and the antisense primer (5′-GCTTGCGTGGCCCCTGGTTCTCA-3′) (SEQ ID NO: 26) anneals at the FCGR2B-specific sequence in intron 6 between exon 6 and 7. The PCR conditions were 94° C. for 2 min, 14 cycles of 98° C. for 20 sec and 68° C. for 17 min, followed by 10 more cycles with the extension time increasing by 15 sec each cycle, and a 7 min extension at 68° C. The resultant 15 kb PCR product was gel-purified and used as the template for the nested-PCR to amplify the 2 kb promoter of FCGR2B with the sense primer (5′-GTTACTCGTTTCTACCTTATC-TTAC-3′) (SEQ ID NO: 27) and the antisense primer (5′-TTGCAGTCAGCCCAGTCACTCTC-3′) (SEQ ID NO: 28). The PCR conditions were 95° C. 5 min, 35 cycles of 94° C. 30 sec, 56° C. 30 sec and 72° C. 2 min, and followed by a 7 min extension at 72° C. The nested-PCR product was then gel-purified and sequenced with BigDye™-terminator cycle sequencing on an ABI 377 (Applied Biosystems, Inc., Foster City, Calif.). The sequencing primer was 5′-ATTTCAAGAAGCATCCAGATTC-3′ (SEQ ID NO: 29). The rare alleles were confirmed by sequencing from both directions.

For genotyping the FCGR2B promoter SNPs, pan-PCR was performed to amplify both FCGR2B/C promoters containing −120 or −386 SNP. For the PCR amplicon of 114 bp containing −120 SNP, the sense primer is 5′-AAAGAGGGTGGAAAGGGAGGAG-3′ (SEQ ID NO: 30) and the antisense primer is 5′-biotin-CTCTCAAAGCTTGGCGGATTCTAC-3′ (SEQ ID NO: 31). For the PCR amplicon containing −386 SNP, the sense primer is 5′-TCAAGAAGCATCCAGAT TCCAG-3′ (SEQ ID NO: 32) and the antisense primer is 5′-biotin-AAACTCAGCTCAGAACCTCCTGTT (SEQ ID NO: 33) -3′. The PCR conditions were 95° C. for 5 min, 40 cycles of 95° C. 30 sec, 56° C. 30 sec and 72° C. 45 sec, followed by a 7 min extension at 72° C. The PCR product was genotyped by pyrosequencing on a PSQ 96 system following the manufacturer's instructions (PyroSequencing AB, Uppsala, Sweden). The pyrosequencing primers for −120 and −386 SNPs were 5′-CCTGTGATAAAACAGAACAT-3′ (SEQ ID NO: 34) and 5′-TGCTGGTGCACGCTGTCCT-3′ (SEQ ID NO: 35) respectively. For the donors who have the uncommon A or C allele at nt −120 or −386, FCGR2B-specific long PCR was then performed to assign the origin of these uncommon alleles by pyrosequencing.

### Transient Transfection and Luciferase Reporter Assays

For BJAB cells, the FCGR2B-promoter reporter plasmid (40 μg) was co-transfected with 300 ng of the reference plasmid pRL-SV40 into 10×10⁶ cells by electroporation at 200 V and 960 μF. For U937 cells, the FCGR2B-promoter reporter plasmid (1 μg) was co-transfected with 100 ng of the reference plasmid into 5×10⁵ cells using 3 μl of FuGENE 6 reagent according to the manufacturer's instructions (Roche Molecular Biochemicals, Indianapolis, Ind.). The cells were recovered overnight and treated with 0.5 mM dibutyryl cAMP, or 400 U/ml IFN-gamma or non-stimulated for additional 24 hr. The cells were then lysed and measured for luciferase activities using the Dual-Luciferase Reporter Assay System (Promega, Madison, Wis.). The firefly luciferase activity was normalized by renilla luciferase activity to yield the relative luciferase activity (RLA).

### Statistical Analysis

Data for comparison of mean values among samples were analyzed by Student's t test or Kruskal-Wallis test. To test for an association between FCGR2B and human SLE, four separate logistic regression models were computed. The four models contained only the FCGR2B haplotype and were sequentially partitioned into 2 degree of freedom tests for general association and three a priori genetic models (i.e., dominant, additive and recessive). The degree of linkage disequilibrium was estimated among FCGR loci using the D and D′ statistics (38). In the joint analysis and the conditional tests of association, to adjust for the effects of FCGR2A and FCGR3A on FCGR2B tests of association, a parallel set of logistic regression models were computed that contain the effects of all three genes with tests of FCGR2B conditional on the FCGR2A/3A genotypes viewed as a priori tests.

### Identification of Single Nucleotide Polymorphisms in the Human FCGR2B Promoter

To identify functional single nucleotide polymorphisms in the human FCGR2B gene, cDNA from more than 120 donors was amplified and sequenced. No non-synonyrnous SNPs in the IgG-binding extracellular domains of FcγRIIb were found (39). However, it was found that the expression levels of FcγRIIb is variable among individuals. Therefore, SNPs were searched for in the regulatory region of FCGR2B gene. Study of polymorphisms in the non-coding regions of FCGR2B is complicated by the extremely high homology between the FCGR2B and FCGR2C genes which reflects gene duplication and cross-over events during evolution of Fc receptor cluster (40-42). To characterize the promoter region of FCGR2B, a BAC library was screened, the FCGR2B and FCGR2C genes were identified, and a 12 kb region of the 5′ portion of each gene was sequenced (42). FCGR2B and FCGR2C are nearly 100% identical within the first 3.4 kb of the 5′ flanking region and regions through exon 3. However, a stretch of 31 nucleotides in the intron 6 (between exons 6 and 7) of FCGR2B is unique to the FCGR2B gene (41, 42). Based on this information, a long-range PCR was developed to specifically amplify the 15 kb of FCGR2B from −2 kb to intron 7 from genomic DNA and a subsequent nested-PCR was also developed using the long PCR product as a template to amplify the FCGR2B promoter for genotyping.

Among 66 non-SLE controls and 66 SLE patients, 10 SNPs were found in the first 2 kb promoter of FCGR2B (FIG. 1). No deletions or insertions were identified in this FCGR2B promoter region. The gene-specificity of the nested PCR strategy was verified by gene-specific SNPs in FCGR2C exon 3 (42).

### Haplotypes in the FCGR2B Proximal Promoter Alter Promoter Activity

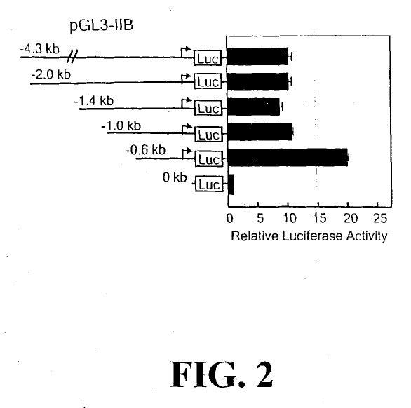

In the mouse, key elements regulating FCGR2B expression are located within the first several hundred bp of the 5′ promoter. To focus on potential functionally important polymorphisms in the human FCGR2B promoter, a series of 5′ deletion promoter-reporter constructs were made and transfected into BJAB cells, a B-lymphoid cell line. Luciferase reporter assays showed that 1.0 kb promoter of FCGR2B retains ≈100% activity as compared with 4.3 kb, 2.0 kb, and 1.4 kb promoter (FIG. 2). There may be a repressor element between −0.6 kb and −1.0 kb because deletion of this promoter fragment leads to a 1.9-fold increase of the luciferase activity (FIG. 2). Similar results were obtained using the same 5′-promoter constructs in U937 cells, a myeloid cell line. Therefore, further study was focused on the 3 SNPs in the proximal 1.0 kb promoter of human FCGR2B.

Among the 132 individuals, the variant “C” allele at nt −386C always was found accompanied by the variant “A” allele at nt −120. The possibility that the −386C and −120A alleles might form a haplotype was confirmed by cloning and sequencing the 1.0 kb FCGR2B promoter from doubly heterozygous donors which identified only two haplotypes, −386G-120T and −386C-120A. The variant G allele at nt −893 (allele frequency is 7%) was about equally represented in both the −386G-120T and −386C-120A haplotypes.

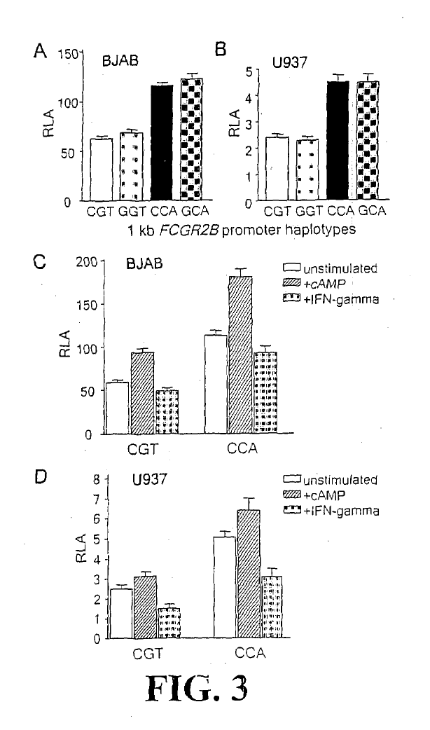

To determine whether the variant alleles affect the promoter activity, luciferase-reporter assays were performed using constructs containing the 1.0 kb FCGR2B promoter incorporating different alleles in front of the luciferase gene. The −893 “C/G” alleles did not influence promoter activity in the context of both −386G-120T and −386C-120A haplotypes in BJAB and U937 cells (FIGS. 3A and B). However, the FCGR2B promoter with the −386C-120A haplotype showed a 1.8-fold greater expression of the luciferase reporter, compared with the −386G-120T haplotype, in both BJAB and U937 cells (FIGS. 3A and B). This difference is apparent in the context of either common “C” or uncommon “G” allele at nt −893. This result showed clearly that the two proximal FCGR2B promoter haplotypes differentially affect constitutive promoter activity.

Recently, several studies have shown that the expression of FcγRIIb is regulated by cytokines and hormones (7, 8, 43-45). Therefore, whether the two FCGR2B promoter haplotypes have differential activity under stimulated conditions was examined. BJAB and U937 cells were transfected with the reporter plasmid containing −893C-386G-120T or −893C-386C-120A haplotype for 16 hours and then stimulated with dibutyryl-cAMP or interferon(IFN)-gamma for 24 hours. In BJAB cells, cAMP upregulated FCGR2B promoter activity by 1.5 fold, and IFN-gamma slightly downregulated FCGR2B promoter in the context of both −386G-120T and −386C-120A haplotypes (FIG. 3C). In U937 cells, for both the FCGR2B promoter haplotypes, cAMP slightly upregulated the promoter activity, and IFN-gamma down-regulated the promoter by 50% (FIG. 3D). These data indicate that the less frequent variant −386C-120A haplotype has greater promoter activity than the more common −386G-120T haplotype under both constitutive and stimulated conditions.

FcγRIIb expression levels on primary cells were also examined, and in agreement with the in vitro luciferase assay presented herein, the donors with the −386C-120A haplotype express more receptor on B lymphocytes and monocytes than donors homozygous for the −386G-120T haplotype (46). The differential promoter activity of the two FCGR2B haplotype is due to their differential binding capacity for transcription factors GATA4 and YY1 (46).

### The Association of FCGR2B Haplotype With SLE

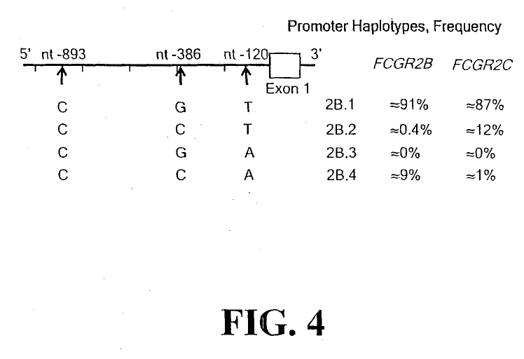

To investigate the relationship of the two functionally important FCGR2B promoter haplotypes to an autoimmune phenotype, a strategy was developed to genotype the two SNPs at nt −386 and −120 in a larger collection of samples. PCR was performed to amplify 114 bp promoter regions containing the −120 SNP of both FCGR2B and FCGR2C genes from genomic DNA. The pan-PCR products were applied to quantitative Pyro-sequencing in a 96 well format which gave 100%, 75%, 50% or 25% allele distributions reflecting the 4 chromosomes from both FCGR2B and FCGR2C genes which were amplified. For the donors with the variant −120A allele, FCGR2B-specific long PCR, followed by nested PCR, was performed and applied to Pyro-sequencing to determine the allele frequency in FCGR2B gene. By this method, the frequency of the variant −120A allele in FCGR2C gene was also determined. A similar strategy was also used for −386 G/C SNPs. For the FCGR2B gene in the Caucasian population, these studies found that the frequency of the common haplotype −386G-120T (named “2B.1” haplotype) is 91%, the uncommon −386C-120A (2B.4) haplotype is approximately 9% (FIG. 4). The −386C-120T (2B.2) haplotype is very rare, and the frequency is about 0.41%. The −386G-120A (2B.3) haplotype in FCGR2B gene has not been observed in the populations utilized for these studies. For the FCGR2C gene, however, the haplotype frequencies are distinct from FCGR2B. The −386C-120T (2B.2) haplotype occurs much more frequently than in FCGR2B gene (12% vs 0.4% haplotype frequency) and the −386C-120A (2B.4) haplotype is much more rare than in FCGR2B gene (1% vs 9%) (FIG. 4). As with FCGR2B, the −386G-120A (2B.3) haplotype has not been observed in the FCGR2C gene. Having established these haplotype frequencies, further association studies were focused on the common −386G-120T and variant −386C-120A haplotype.

In the collection of 243 Caucasian SLE patients and 366 matched controls utilized in these studies, the less frequent variant 2B.4 (−386C-120A) haplotype in the FCGR2B gene promoter was significantly associated with the autoimmune SLE phenotype (Table 2, single locus association test using logistic regression analyses, additive model, P=0.0054, odds ratio=1.65, 95% confidence interval=1.16-2.36). Unlike FCGR2B, there was no association of FCGR2C alleles or haplotypes with SLE (P=0.975).

TABLE 2
Distribution of the FCGR2B promoter haplotypes in SLE
patients and controls*

| | Caucasian | Caucasian | |
|---|---|---|---|
| | Controls | Patients | |
| | n = 366 | n = 243 | SLE |
| | No. of subjects (% of group) | | |
| Genotype | | | |
| 2B.1/2B.1 | 300 (82.0%) | 180 (74.1%) | |
| 2B.1/2B.4 | 63 (17.2%) | 56 (23.0%) | |
| 2B.4/2B.4 | 3 (0.8%) | 7 (2.9%) | |
| Haplotype Frequency | | | |
| 2B.1 (−386G-120T) | 90.6% | 85.6% | |
| 2B.4 (−386C-120A) | 9.4% | 14.4% | |

*Haplotype frequency in SLE patients vs. controls (2 × 2 Chi-square test, OR = 1.62, P = 0.007; logistic regression analyses, additive model, odds ratio = 1.65, P = 0.0054, 95% confidence interval = 1.16-2.36).

The association of FCGR2B promoter haplotype with SLE was not due to the presence of an extended haplotype containing the recently reported non-synonymous exon 5 SNP which encodes a transmembrane polymorphism and which associates with the SLE phenotype in a Japanese population (47). The uncommon transmembrane allele, 775T→C encoding Ile¹⁸⁷→Thr¹⁸⁷, was found in only several donors with the 2B.4 promoter haplotype and the variant Thr¹⁸⁷⁷ is not associated with SLE in Caucasian population (39).

### Combined Analysis of FCGR2B, FCGR2A, FCGR3A Polymorphisms

Functional polymorphisms in the extracellular domains of FcγRIIa and FcγRIIIa have been shown to associate with SLE in a number of studies (26). Since FCGR2B is located about 200 kb telomeric to FCGR2A/3A within the classical Fc receptor cluster, the potential linkage disequilibrium among the polymorphisms of these three genes in the collection of Caucasians was examined. Analyses of the SNP genotyping data show that there is no linkage disequilibrium of FCGR2B promoter haplotypes with FCGR2A and FCGR3A polymorphisms since the calculated D′ was low (D′=0.221 and D′=0.486 respectively) and there is no statistical interaction between FCGR2B and FCGR3A loci under a dominant-additive genetic model (P=0.6629). The three conditional association tests for FCGR2A, FCGR3A and FCGR2B polymorphisms were also computed. Logistic regression, adjusted for FCGR2A and FCGR3A, showed a persistent effect for FCGR2B (Table 3, P=0.0083, odds ratio=1.72, 95% confidence interval=1.15-2.58). After adjusting for FCGR2B polymorphisms, FCGR3A is also significantly associated with SLE (Table 3, P=0.0288, odds ratio=0.65, 95% confidence interval=0.44-0.96). This lack of substantial linkage disequilibrium from FCGR2B to FCGR2A/3A is consistent with the physical distance of 250 kb across this cluster which is larger than the median haplotype block in Caucasians (42, 48). Therefore, FCGR2B and FCGR3A may contribute to SLE independently and, perhaps, synergistically.

TABLE 3
Joint analysis and the conditional tests of association

| | General Test of | Best Genetic Model | OR (95% CI) |
|---|---|---|---|
| | Association | Test of Association | Under Best |
| | (P value) | (P value) | Genetic Model |
| FCGR2A | 0.9929 | Dominant, 0.9720 | 1.01 (0.61-1.67) |
| FCGR3A | 0.0949 | Dominant, 0.0288 | 0.65 (0.44-0.96) |
| FCGR2B | 0.0204 | Additive, 0.0083 | 1.72 (1.15-2.58) |

The ITIM-bearing IgG receptor FcγRIIb is widely expressed on immune cells and plays an important role in maintaining immune response homeostasis. FCGR2B-deficient mice have elevated immunoglobulin levels in response to both thymus dependent and independent antigens and, on a susceptible genetic background, FCGR2B-deficient mice develop a lupus-like autoimmune disease (20, 21). Polymorphisms in the mouse homolog of the human FCGR2B gene have been identified in several autoimmune-prone strains (14, 16). Taken together, these observations have focused attention on FcγRIIb both as a disease susceptibility gene and as a potential therapeutic target for autoimmunity.

To assess the role of FcγRIIb in human autoimmunity, the functional genetic variations in FCGR2B gene were identified and their association with the SLE phenotype was assessed. The two FCGR2B proximal promoter haplotypes which were found in more than 99% of all 600 donors studied were characterized. The two FCGR2B haplotypes have differential promoter activity in cell lines of lymphoid and myeloid lineages under both constitutive and stimulated conditions. The less frequent, variant gain-of-function promoter haplotype of FCGR2B is significantly enriched in SLE patients in our case-control study of Caucasians with an odds ratio 1.65. This disease association is not due to linkage disequilibrium with other Fc receptor family genes (FCGR2A or FCGR3A).

The association of the gain-of-function promoter variant of FCGR2B with human SLE might be considered a surprise. However, the effect of the homozygous FCGR2B knock-out mouse is background dependent, and the repertoire of Fc receptors in mouse is different from that in humans. Mouse has a single CD32 gene (the ITIM-containing FCGR2B) while humans have two additional CD32 genes, the ITAM-containing activation receptors, FCGR2A and FCGR2C. FcγRIIb is expressed on multiple cell types and may have distinct function(s) depending on its cell context. For example, expressed on mononuclear phagocytes, FcγRIIb can decrease the phagocytosis of immune complexes, a process important for the in vivo clearance of immune complex. On follicular dendritic cells (FDC), FcγRIIb promotes the maturation of FDC reticulum and mediates the uptake and conversion of immune complexes on FDCs to potentially more highly immunogenic forms (10-13, 49). In contrast on B cells, FcγRIIb downmodulates B cell activation and antibody production. Thus, FcγRIIb may play distinct roles according to the disease stage and the cell types involved in the development of autoimmunity. In considering modulation of FcγRIIb as a therapeutic target, it may be important to consider cell-type specific targeting according to the disease characteristics and its developmental stage.

The association of the C-A promoter haplotype with the SLE phenotype suggests human FCGR2B as a candidate gene for autoimmune susceptibility. Human SLE is a complex, polygenic genetic trait with a strong genetic component (26). Four independent genome wide scans support linkage of chromosome 1q21-23 which encompasses the FcγR cluster with systemic lupus erythematosus (50-53). SNPs in FCGR2A and FCGR3A have shown linkage and association with SLE in both family-based and case-control based studies (odds ratio 1.5 to 2.2 for FCGR3A) (28-32). According to the linkage disequilibrium analysis and conditional tests of association within the FcγR cluster, the association of FCGR2B with SLE does not represent disequilibrium with FCGR2A or FCGR3A. FCGR2B and FCGR3A contribute to autoimmunity independently.

The data presented herein demonstrate the occurrence of two functionally distinct FCGR2B promoter haplotypes which affect promoter activity in both lymphoid and myeloid cell lines. The two FCGR2B promoter haplotypes have differential binding capacity for transcription factors GATA4 and YY1 and lead to differential expression levels of the endogenous FcγRIIb on primary cells (46). Identification of the FCGR2B promoter variants as a disease risk factor also supports the notion that duplicated regions within the genome are likely the hot spots of genomic instability and are associated with genetic diseases (55). Furthermore, apart from autoimmunity, FCGR2B promoter genotypes may also play an important role in the variations of human antibody responses to vaccines as predicted by its function on B cells and studies in the mouse (56).

### EXAMPLE II

### A Promoter Haplotype of the ITIM-Bearing FcγRIIb Alters Receptor Expression and Associates With Autoimmunity. II. Differential Binding of GATA4 and YY1 Transcription Factors and Correlated Receptor Expression and Function¹

The ITIM-containing FcγRIIb modulates immune function on multiple cell types including B cells, monocytes/macrophages, and dendritic cells. The promoter for the human FCGR2B is polymorphic and the less frequent 2B.4 promoter haplotype is associated with the autoimmune phenotype of systemic lupus erythematosus. In the present study, it was demonstrated that the 2B.4 promoter haplotype of FCGR2B has increased binding capacity for GATA4 and YY 1 transcription factors in both B lymphocytes and monocytes, and that overexpression of GATA4 or YY1 enhances the FCGR2B promoter activity. The 2B.4 haplotype leads to elevated expression of the endogenous receptor in heterozygous donors by 1.5 fold as assessed on EBV-transformed cells, primary B-lymphocytes and CD14⁺ monocytes. This increased expression accentuates the inhibitory effect of FcγRIIb on B cell antigen receptor signaling, measured by Ca²⁺ influx and cell viability in B cells. Our results indicate that transcription factors GATA4 and YY 1 are involved in the regulation of FcγRIIb expression and that the expression variants of FcγRIIb lead to altered cell signaling, which may contribute to autoimmune pathogenesis in humans.

The IgG Fc receptors play an important role in regulating immune system by bridging the humoral and cellular immune responses (2, 3, 33, 57). On mouse follicular dendritic cells (FDC), FcγRIIb is the highly expressed IgG Fc receptor and can mediate the retention and conversion of immune complexes on FDCs to a highly immunogenic form (10, 11) which may play a role in affinity maturation and memory B cell development (12, 13). Similarly, on Langerhans cells, FcγRIIb mediates antigen internalization and presentation (58-60). On B cells, at least in part by recruitment of phosphatases to its immuno-receptor inhibitory motif (ITIM), FcγRIIb engagement can shape the antibody repertoire through modulation of BCR-mediated cell activation and proliferation (5, 6), through signals for apoptosis independent of BCR (61) and through down-regulation of pre-B cell antigen receptor (BCR)-mediated apoptosis (62). On myeloid lineage cells, FcγRIIb downregulates antibody-mediated phagocytosis and inflammatory responses when clustered with the activating Fcy receptors, such as FcγRIa, FcγRIIa, and FcγRIIIa (7, 9). Thus through its roles in facilitating antigen presentation and in regulating B cell survival and proliferation, FcγRIIb has a significant role in maintaining immune homeostasis, which makes FcγRIIb an attractive functional candidate for autoimmune diseases.

As stated above, this invention has demonstrated that a functional promoter haplotype in the human FCGR2B gene is associated with systemic lupus erythematosus (SLE) (63), suggesting that FcγRIIb contributes to susceptibility for autoimmune disease. To address the underlining molecular mechanism in relation to the in vivo function of these FCGR2B haplotypes, the transcription factor-binding capability of the polymorphic sites within the FCGR2B promoter haplotypes were explored. Computer-based searches suggested that the single nucleotide polymorphisms (SNPs) were located in putative GATA family and YYI transcription factor binding elements. Direct assessment of binding indicated that the allelic variants from the less frequent 2B.4 haplotype have increased binding capacity for both GATA4 and YY1 transcription factors in B lymphocytes and monocytes. Overexpression of either GATA4 or YY1 upregulates FcγRIIb promoter activity, suggesting that GATA4 and YY1 are involved in the regulation of FcγRIIb expression. Among genotyped donors, the 2B.4 haplotype leads to higher expression of endogenous FcγRIIb on both primary B-lymphocytes and monocytes. This increased receptor expression accentuates the FcγRIIb function as measured by BCR-induced Ca²⁺ influx and cell viability in B cells. Thus, our data indicate that the FCGR2B promoter SNPs occur in transcription factor binding elements and alter transcription factor binding, that GATA4 and YY1 transcription factors regulate FcγRIIb expression, and that the resultant change in expression can alter cell function. Given the several roles that FcγRIIb may play in the pathogenesis of autoimmunity, the specific function for FcγRIIb may vary according to the nature and stage of the disease.

### Donors

Caucasian SLE patients and disease-free controls were recruited as part of the University of Alabama at Birmingham-based DISCOVERY cohort, a population-based case-control study. The studies were reviewed and approved by the Institution Review Board, and all donors provided written informed consent.

### Reagents

AT-10-FITC was purchased from Serotec Inc. (Raleigh, N.C.). The IV.3 hybridoma cell line was purchased from ATCC, and purified IV.3 antibody was conjugated with FITC with FITC-labeling kit (Sigma, St. Louis, Mo.). Anti-CD19-APC, anti-CD14-TRI-COLOR, anti-CD56-PE and anti-CD3-PE mAb were purchased from Caltag Laboratories (Burlingame, Calif.). The FcγRIIb-specific polyclonal antibody was generated by immunization of rabbits with GST fusion protein containing the unique cytoplasmic domain of FcγRIIb. Goat anti-FcγRIIa/c polyclonal antibody, anti-YY1, anti-GATA1, 2, 3, 4, and 6 antibodies were purchased from Santa Cruz Biotechnogy (Santa Cruz, Calif.). Anti-HisG and anti-Xpress tag antibodies were purchased from Invitrogen (Carlsbad, Calif.). The A20-IIA1.6 cell line was kindly provided by Dr. Terri Wade at Dartmouth Medical Center (39).

Flow cytometry was performed using mAb IV.3 and AT-10 to compare their staining patterns on FcγRIIa- or FcγRIIb transfectants. For FcγRIIb, mAb AT-10 stains about 10 times stronger than mAb IV.3. In contrast, for both FcγRIIa alleles (H131 and R131), mAbs AT-10 and IV.3 have comparable reactivity (less than 2-fold difference between the two mAbs). Thus, mAb IV.3 weakly recognizes FcγRIIb when highly expressed in transfected cell lines.

### Plasmid Construction

For luciferase-based constructs, various human FcγRIIb promoter fragments were amplified by PCR from genomic DNA and subcloned into the luciferase reporter vector pGL3-Basic (Promega). The alternative alleles were introduced at the polymorphic sites of the FcγRIIb promoter using QuickChange site-directed mutagenesis (Stratagene). For the mammalian expression of GATA4 and YY1 transcription factors, the cDNA of GATA4 or YY1 was amplified by RT-PCR from BJAB and Hela cells respectively and subcloned into pcDNA3His expression vector (Invitrogen). The expressed protein was N-terminally tagged with His₆Gly and Xpress epitopes. For transient expression of human FcγRIIa (both H131 and R131 alleles), the cDNA was amplified by RT-PCR from peripheral mononuclear cells isolated from whole blood of an FcγRIIa H131/R131 heterozygous donor. The FcγRIIa cDNA was subcloned into pcDNA3His vector for expression in Cos-7 cells (see below). All the constructs were confirmed by direct DNA sequencing.

The PCR primers for the cloning of human GATA4 cDNA were: sense, 5′-GCAGGTACCCATGTATCAGAGCTTGGCCATG-3′ (SEQ ID NO: 36); anti-sense, 5′-GAAGAATTCAGATTACGCAGTGATTATGTCCC-3′ (SEQ ID NO: 37). The PCR primers for the cloning of human YY1 cDNA were: sense, 5′-CGCGGATCCACCATGGCCTCGGGCGACACC (SEQ ID NO: 38); anti-sense, 5′-CGGAATTCTCACTGGTTGTTTTTGGCCTTAG-3′ (SEQ ID NO: 39). The PCR primers for the cloning of human FcγRIIa cDNA were: sense, 5′-CGGAATTCATGGCTATGGAG ACCCAAATGTC-3′ (SEQ ID NO: 40); anti-sense, 5′-CTGTCTAGATTAGTTATTACTGTTGACATG GTCG-3′ (SEQ ID NO: 41).

### Reverse Transcription Polymerase Chain Reaction (RT-PCR)

The total RNA was prepared from different types of cells using Trizol Reagents (Invitrogen/GIBCO BRL). The cDNAs were synthesized using SuperScript™ Preamplification System (Invitrogen/GIBCO BRL). The gene-specific PCR reaction was performed in a 9600 PCR System with 2 μl of cDNA, 200 nM of each primer, and 2.5 U of DNA polymerase from Failsafe PCR system (Epicenter Technologies, Madison, Wis.) starting with 94° C. for 2 min, 28 cycles of denaturing at 98° C. for 20 sec, annealing at 58° C. for 30 sec, and extension at 68° C. for 90 sec with a final extension at 68° C. for 7 min. The PCR product was purified using QIAquick Gel Extraction Kit (QIAGEN Inc., Chatsworth, Calif.).

### Electrophoretic Mobility Shift Assays (EMSA).

The oligonucleotide probes for EMSA were labeled by Klenow fill-in with α-³²P-dCTP. Nuclear extracts were prepared using NE-PER nuclear and cytoplasmic extraction reagents (Pierce, Rockford, Ill.). EMSA was performed with 6 μg of nuclear extract and 20,000 cpm ³²P-labeled probe in 20 μl of binding buffer (10 mM Hepes (pH 7.5), 50 mM KCl, 5% glycerol, 2 mM MgCl₂, 0.2 mM EDTA, 0.2 mg/ml BSA, 1 μg of polydeoxyinosinic-deoxycytidylic acid, 1 mM DTT, 1 mM Pefabloc). The labeled probe was incubated with nuclear extract at room temperature for 20 min. Bound and free DNA probe were then resolved by electrophoresis through a 6% polyacrylamide gel in 0.5× Tris-borate-EDTA buffer at 200 volts for 2 h. The gel was dried and exposed to film for autoradiography. For competition and super-shift assays, prior to the addition of the labeled probe, a 200-fold molar excess of the indicated unlabeled oligonucleotides or 4 μg of antibodies were added to the nuclear extracts and incubated at 4° C. for 1 hr. The labeled probe was then added and incubated at room temperature for additional 20 min followed by electrophoresis.

### Transient Transfections

For luciferase assays, reporter plasmid pGL-2B (10 μg) was co-transfected with the reference plasmid pRL-SV40 (150 ng) and the GATA4 or YY1 expression vector pcDNA (1 μg) into 10×10⁶ BJAB cells by electroporation at 200 V and 960 μF. For U937 cells, reporter plasmid pGL-2B (0.5 μg) was co-transfected with the reference plasmid pRL-SV40 (30 ng) and the GATA4 or YY1 expression vector pcDNA (50 ng) into 5×10⁵ U937 cells in 12 well plates using 1.5 μl of FuGENE 6 reagent according to the manufacturer's instructions (Roche Molecular Biochemicals, Indianapolis, Ind.). The luciferase activities were measured at 40 hr after transfection using the Dual-Luciferase Reporter Assay System (Promega, Madison, Wis.). The firefly luciferase activity was normalized by renilla luciferase activity to yield the relative luciferase activity (RLA).

For Cos-7 transfections, cells (60-80% confluent) in 10 cm plates were transfected with 6 μg of plasmids and 18 μl of Fugene 6 reagent according to the manufacturer's instructions. Cells were harvested for preparation of nuclear extracts or whole cell lysate at 30 hr post transfection.

### Preparation of Whole Cell Lysate and Immunoprecipitation Assay.

Cells were lysed with whole cell lysis buffer (19) at 20 μl/1×10⁶ cells for EBV cells and monocytes, or 60 μl/1×10⁶ cells for Cos-7 and A20-IIA1.6-FcγRIIb transfectants (19). The samples were vortexed for 10 sec and incubated on ice for 30 min with a brief vortexing every 10 min. The samples were then centrifuged at 15,000 rpm at 4° C. for 15 min and the supernant was collected.

For immunoprecipitation, mAbs 32.2, IV.3 or AT-10 were added to the whole cell lysate and incubated at 4° C. for 2 h with mixing. Protein G Sepharose beads were added to each sample and the samples were further incubated at 4° C. for 1 h with mixing. The beads were washed 4 times with whole cell lysis buffer and the immunoprecipitates were subjected to western blot analysis.

Purification of CD14⁺ Monocytes from Whole Blood.

Peripheral blood mononuclear cells (PBMCs) were isolated by density gradient centrifugation using Ficoll Hypaque followed by CD14 Magnetic MicroBeads (Miltenyi Biotec Inc. Auburn, Calif.). The CD14⁺ monocytes were purified on positive selection columns (MS⁺). Multicolor flow cytometry (anti-CD19-APC for B lymphocytes, anti-CD3-TRI-COLOR for T lymphocytes, anti-CD56-PE for NK cells, and mAb IV.3-FITC for monocytes) was performed on the separated cell populations to determine the purity (>90%) and recovery (50-70%).

### Measurement of Change in [Ca²⁺]ᵢ

Changes in intracellular [Ca²⁺]ᵢ induced by cross-linking of surface Ig on EBV-transformed B cells were determined using an SLM 8000 spectrofluorometer monitering the simultaneous 405/490 nm fluorescence emission ratio of the calcium binding indo-1 fluorophore, as previous described (29). Cells (10×10⁶/ml) were loaded with 5 μM of indo-1-AM at 37° C. for 40 min and stimulated with 10 μg/ml goat IgG anti-human κ or an equal-molar concentration of goat F(ab)′₂ anti-human K (Southern Biotechnology Associates, Birmingham, Ala.) at the 60 sec time point.

### Cell Viability Assay

EBV-transformed B cell lines from genotyped donors were treated with 6.7 μg/ml goat F(ab)′₂ anti-human IgM or 10 μg/ml goat IgG anti-human IgM for 60 hours. The ATPlite assay was performed in 96 well assay plates with 200 cells/well and each condition was performed in triplicate. The cells were lysed and assayed for the amount of ATP on a Packard TopCount Microplate Scintillation and Luminescence Counter following the manufacturer's direction (Packard, Meriden, Conn.).

SNPs on the 2B.4 Haplotype have Increased Binding Capacity for GATA4 and Yin-Yang1 Transcription Factors

This invention provides two functional haplotypes (−386G-120T and −386C-120A) in the proximal promoter region of human FCGR2B gene (63). Case-control studies have suggested that the gain-of-function 2B.4 haplotype (−386C-120A) is associated with SLE phenotype (63). To explore the molecular basis for the differential function of the two promoter haplotypes, we performed electrophoretic mobility shift assays (EMSA) to determine the capability of −120 T/A SNP and −386 G/C SNP regions to bind with transcription factors.

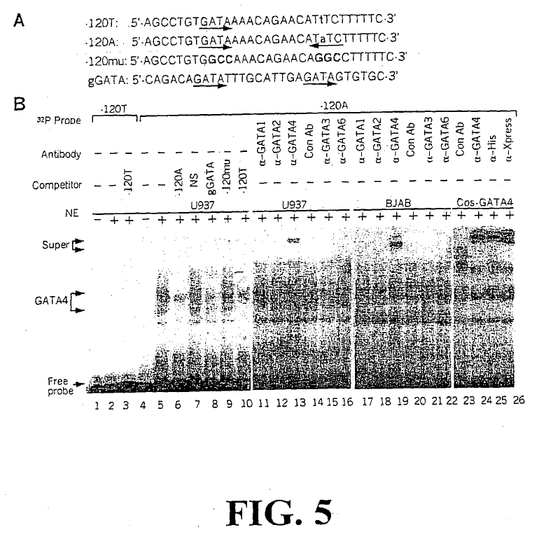

Computer-based searches revealed that a GATA-binding motif is located 12-15 nucleotides 5′ to the −120T/A SNP and that the −120A allele creates a second GATA-binding motif, thus forming palindromic binding sites for GATA (FIG. 5A). EMSAs using U937 nuclear extracts showed that a −120A probe had a much higher binding capacity for transcription factors than a −120T probe (FIG. 5B, lanes 1-5). A 200-fold excessive of unlabeled −120A oligonucleotides, but not non-specific oligonucleotides, effectively blocked binding of labeled probe (FIG. 5B, lanes 6 and 7). The protein binding to the −120A probe was partially competed away by unlabeled GATA1-binding oligonucleotides derived from human γ-globin promoter (64), but −120 oligonucleotides with “GATA” motif mutated were ineffective in competition experiments (FIG. 5B, lanes 8 and 9). These data suggested that the transcription factor was a GATA family member. Unlabeled −120T oligonucleotides (containing only one “GATA”-motif) less efficiently competed the binding consistent with its lower binding capacity for the transcription factor relative to the −120A oligonucleotides (FIG. 5B, lane 10).

Because there are six known members of the GATA family of transcription factors, a panel of GATA-specific antibodies was used for super-shift assays to determine which GATA member binds to −120A probe. Anti-GATA4 antibody resulted in a super-shift of the complexes while neither a control antibody nor other anti-GATA family member antibodies affected the binding (FIG. 5B, lanes 11-16). These data suggest that the transcription factor bound to the −120A probe is GATA4 in monocytic U937 cells. Similar differential binding capacity for a GATA transcription factor on −120T/A probes was also obtained with nuclear extracts from BJAB cells. Super-shift experiments indicate that in BJAB cells GATA4 is also the predominant GATA species, which binds to −120A, probe (FIG. 5B, lanes 17-22).

To further demonstrate that −120T/A alleles have differential binding capacity for the GATA4 transcription factor, human GATA4 was transiently overexpressed in Cos-7 cells using the pcDNA3His vector. EMSAs using nuclear extracts from these transfectants showed that −120A probe has increased binding capacity for GATA4 compared to the −120T probe and antibodies against GATA4, His₆G and Xpress tags all super-shifted the binding complex (FIG. 5B, lanes 23-26). Taken together, these data suggest that the variant −120A allele has increased binding capacity for GATA4 transcription factor in both B and monocyte cell lines and provide additional evidence that palindromic GATA-binding motifs have much higher binding capacity for the transcription factor than a single GATA-binding site (64).

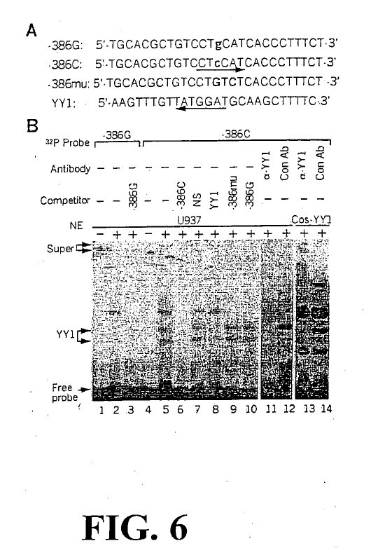

For −386 G/C SNP, computer-based searches revealed that the less frequent allele −386C created a binding motif for a universal transcription factor Yin-Yang1 (YY1) (FIG. 6A). EMSA experiments showed that two DNA-protein complexes formed only on −386C probe but not at detectable level on −386G probe (FIG. 6B, lanes 1-5, indicated by “YY1” double arrows). Those binding were specific because unlabeled −386C oligonucleotides, but not by non-specific oligonucleotides, effectively competed for binding (FIG. 6B, lanes 6 and 7). Binding was also blocked by known YY1-binding oligonucleotides “YY1” (derived from human gp91^{phox} gene promoter (65)) but not by −386 mutant oligonucleotides with three nucleotides critical for YY1 binding mutated or by −386G oligonucleotides containing no YY1 binding motif (FIG. 6B, lanes 8-10). These data suggested that the complexes contain YY1.

Super-shift experiments using anti-YY1 specific antibodies indicated that both protein complexes contain YY1 (FIG. 6B, lanes 11 and 12). Overexpression of human YY1 in Cos-7 cells confirmed the differential YY1 binding on −386 G/C alleles (FIG. 6B, lanes 13 and 14). Another as yet unidentified transcription factor binds similarly on both −386G and −386C probes (the third band above the YY1 bands, FIG. 6B, lanes 2 and 5), suggesting that the polymorphic −386 alleles are not critical for this transcription factor binding. Similar differential binding for YY1 transcription factor was also obtained using BJAB nuclear extracts. Taken together, these data suggest that the variant −386C allele has much higher binding capacity for YY1 transcription factor than the −386G allele in both U937 monocytes and BJAB B cells.

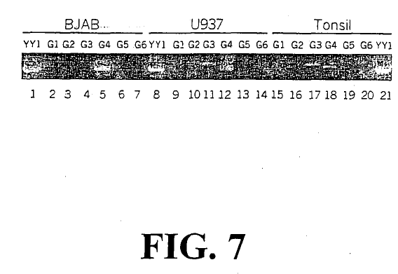

To further support the EMSA and super-shift data provided herein, RT-PCR was performed to confirm the expression of GATA4 and YY1 transcription factors in BJAB and U937 cells. Gene specific RT-PCR for YY1 and each GATA family member demonstrated that YY1 is universally expressed and GATA4 is the predominant GATA family member expressed in BJAB cells (FIG. 7). U937 cells express GATA4 and, to a lesser extent, GATA3. Primary tonsillar cells express both GATA3 and GATA4 (FIG. 7).

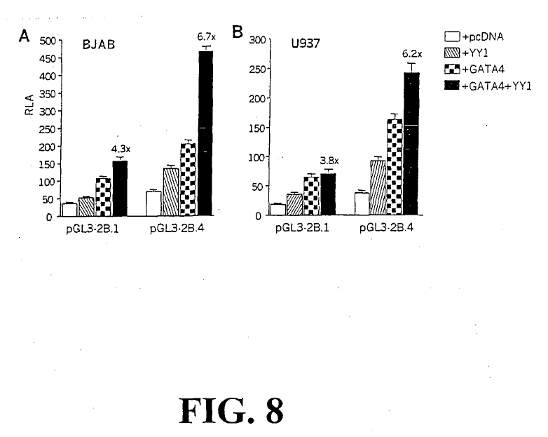

To demonstrate that GATA4 and YY1 bind to the FCGR2B promoter in vivo and are involved in the transcriptional regulation of FCGR2B, we tested whether overexpression of those transcription factors will affect the promoter activity of the FCGR2B gene. Co-transfection of the FCGR2B promoter reporter plasmids (1 kb FCGR2B promoter was placed in front of the luciferase gene) with the pcDNA3-GATA4 and/or YY1 expression vector demonstrated that overexpression of GATA4 or YY1 enhanced the luciferase expression by 2 to 3-fold in BJAB and U937 cells (FIGS. 8A and B). Overexpression of GATA4 and YY1 synergistically leads to about 6-fold increase of the luciferase expression in the context of the variant 2B.4 (−386C-120A) haplotype while about 4-fold increase in the context of the low-binding 2B.1 (−386G-120T) haplotype. Our data demonstrate that transcription factors GATA4 and YY1 are involved in the regulation of FCGR2B promoter in vivo and the variant 2B.4 haplotype has an increased capacity to respond to those transcription factors.

### 2B.4 Haplotype Leads to Elevated FcγRIIb Expression on B Lymphocytes

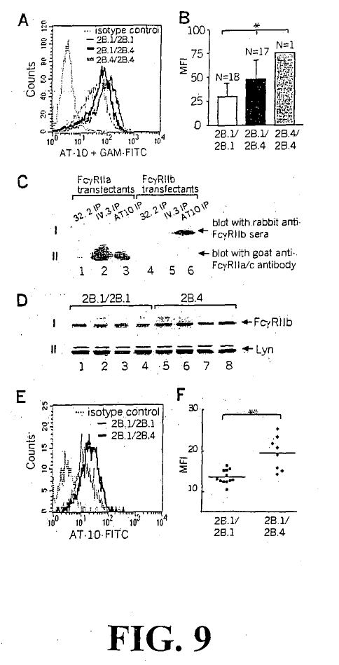

Recognizing that the 2B.1 and 2B.4 haplotypes occur naturally in human donors, possible differential expression levels of FcγRIIb were explored of both transformed cells and primary cells ex vivo. mAb AT-10 was used in flow cytometry to determine the FcγRIIb expression levels on B cells. Binding of mAb IV.3 to both EBV-transformed B cells and peripheral B lymphocytes was indistinguishable from that of isotype control suggesting that these B cells do not express detectable levels of FcγRIIa in agreement with others (57, 66). Using EBV-transformed B cells from 18 2B.1/2B.1 homozygous, 17 2B.1/2B.4 heterozygous, and 1 2B.4/2B.4 homozygous donors, it was demonstrated that the 2B.1/2B.4 heterozygous donors had 1.5-fold increased FcγRIib expression on EBV-B cells and the single 2B.4 homozygous donor had 2.5-fold increased FcγRIIb expression compared to the 2B.1 homozygous donors (FIGS. 9A and B). Furthermore, a rabbit polyclonal antibody was developed that specifically recognizes the unique cytoplasmic domain of FcγRIIb. The present invention shows that both mAb IV.3 and AT-10 immunoprecipitates from Cos 7-FcγRIIa transient transfectants were not recognized by our rabbit anti-FcγRIIb sera (FIG. 9C, panel I, lanes 1-3), however, they were recognized by a goat-anti-FcγRIIa/c cytoplasmic domain antibody (FIG. 9C, panel II, lanes 1-3). The mAb AT-10 immunoprecipitates from A20-IIA1.6-FcγRIIb stable transfectants (39) were recognized by rabbit anti-FcγRIIb sera (FIG. 9C, panel I, lane 6), but not by goat anti-FcγRIIa/c antibody (FIG. 9C, panel II, lane 6). These data demonstrate that the rabbit anti-FcγRIIb antibody is FcγRIIb-specific and can be used to specifically detect the expression of FcγRIIb by western blot. Western blot analysis using the FcγRIIb -specific anti-sera, after normalization for protein loading with an anti-Lyn antibody, detected a 2.3-fold increased expression levels of FcγRIIb in the EBV-transformed cells from the 2B.4-containing donors compared to that from the 2B.1 homozygous donors (FIG. 9D).

Freshly explanted peripheral blood B-lymphocytes were also studied ex vivo by multicolor flow cytometry. Staining by mAb AT-10 on CD19⁺ peripheral B lymphocytes from 12 homozygous 2B.1/2B.1 and 8 heterozygous 2B.1/2B.4 normal donors, showed a 1.4-fold higher expression of surface FcγRIIb from 2B.1/2B.4 heterozygotes relative to 2B.1/2B.1 homozygotes (FIGS. 9E and F). The difference in the expression levels of FcγRIIb is comparable to that seen with EBV-transformed B cells.

### 2B.4 Haplotype Leads to Higher Expression of FcγRIIb on CD14⁺ Monocytes

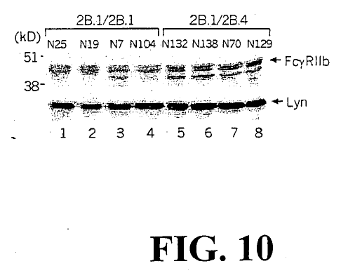

Since the differential promoter activity of the two haplotypes is evident in both B and monocytic cell lines, FcγRIIb expression on freshly isolated monocytes from genotyped non-SLE normal donors was examined. High levels of FcγRIIa expression on CD14⁺ monocytes precluded the ability to detect any expression difference of FcγRIIb by mAb AT-10 using flow cytometry. Therefore, to determine the FcγRIIb expression levels on peripheral monocytes, CD14⁺ monocytes were purified from Ficoll-separated mixed mononuclear cell by anti-CD14 Magnetic MicroBeads. Multicolor flow cytometry was performed on the separated cell populations to confirm the purity (>90%) of the monocytes using markers for B (CD19) and T (CD3) lymphocytes, NK cells (CD56), and monocytes (IV.3). The purified monocytes were lysed and equal amount of whole cell lysate was applied to western blot analysis using the FcγRIIb-specific polyclonal antibody. The data showed elevated FcγRIIb expression on peripheral monocytes from 2B.1/2B.4 heterozygous donors compared to 2B.1/2B.1 homozygous donors (FIG. 10).

Differential Inhibitory Function of FcγRIIB from Genotyped Donors

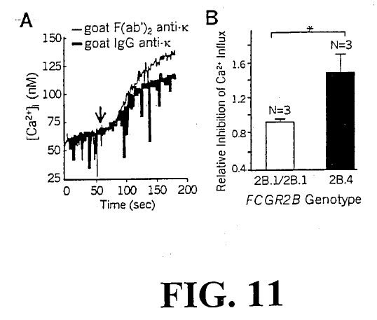

To determine whether the differential FcγRIIb expression among the donors could have differential inhibitory effects, BCR-induced intracellular Ca²⁺ fluxes by F(ab)′₂ or whole IgG anti-γ stimulation were assayed. EBV-transformed cell lines from 3 2B.1/2B.1 and 3 2B.4-containing donors (2 2B.1/2B.4 heterozygous and 1 2B.4 homozygous donor) were used. Ca²⁺ influx was induced by engagement of BCR and downregulated by co-engagement of BCR and FcγRIIb (FIG. 11A). The FcγRIIb from the 2B.4-containing donors had 1.5-fold higher inhibitory effects on the BCR-mediated Ca²⁺ influx than that from the 2B. 1 homozygous donors (FIG. 11B).

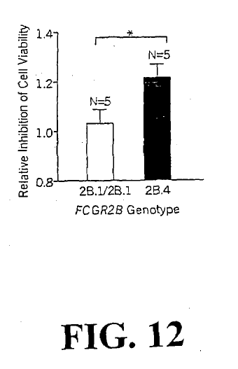

The potential differential function of FcγRIIb on B cell proliferation and viability was examined next. EBV cells from 5 homozygous 2B.1/2B.1 and 5 2B.4-containing (4 heterozygous 2B.1/2B.4 and 1 homozygous 2B.4/2B.4) donors were stimulated with F(ab)′₂ or whole IgG anti-IgM for about 60 hours and the ATP-lite assay was performed to measure the cell viability. F(ab)′₂ anti-human IgM stimulation led to decreased cell viability by 30-80% compared with untreated controls. Co-engagement of anti-BCR and FcγRIIb inhibited this effect and FcγRIIb from 2B.4-containing donors showed 20% more inhibition than that from 2B.1 donors (FIG. 12). Thus, the data demonstrate that the naturally occurring FcγRIIb expression variants differentially impact B cell activation and viability.

Many studies have suggested that FcγRIIb, the ITIM-containing IgG receptor expressed on B lymphocytes and myeloid lineage cells, plays an important role in maintaining immune homeostasis and tolerance (2, 3). In mouse models, complete targeted disruption of FcγRIIb on the susceptible C57B6 background leads to expression of lupus-like phenotypes (20, 21). Among inbred mouse strains, a haplotype affecting FcγRIIb expression is present in many autoimmune strains (14, 16). These observations have implicated FcγRIIb as a potential susceptibility gene for autoimmunity.

The present invention provides genetic variations affecting FcγRIIb expression and function in humans. The present invention has also shown the association of the lower frequent 2B.4 FCGR2B promoter haplotype with SLE (63). In this study, the two promoter haplotypes of human FCGR2B gene that differ in both their in vitro and ex vivo activities were further characterized. The 2B.4 FCGR2B promoter haplotype has an increased binding capacity for GATA4 and YY1 transcription factors compared to the more frequent 2B.1 haplotype. Donors with one copy of 2B.4 haplotype have ≈1.5-fold elevated FcγRIIb receptor expression compared to 2B.1 homozygous donors when assessed on EBV-transformed cells, fresh peripheral B lymphocytes and CD14⁺ monocytes. The FcγRIIb from 2B.4-containing donors has accentuated inhibitory function compared to that from 2B.1 donors on BCR-induced Ca²⁺ influx and on cell viability. Such differences could have significant biological consequences in vivo. For example, subtle differences in BCR function, modulated by 20% differential CD19 expression, have a strong influence on the development of an autoimmune phenotype (67).

GATA4 and the universally expressed YY1 are responsible for the differential promoter activity of the FCGR2B haplotypes in B cells and monocytes. In vitro EMSA assays demonstrate that the variant −120A allele has increased binding capacity for GATA4 and that the variant −386C has increased binding capacity for YY1. Over-expression of YY1 and GATA4 enhances the FCGR2B promoter activity, and the enhancement is more dramatic with the 2B.4 promoter haplotype than the common haplotype. The increased luciferase reporter expression by YY1 and/or GATA4 in the context of the low-binding 2B.1 haplotype, although lower than the 2B.4 haplotype, was more than expected. While this may reflect the stoichiometry and mass action of over-expressed transcription factors binding to the polymorphic site, we searched for additional potential binding elements for GATA and YY1 within the 1 kb promoter region of FCGR2B. In addition to the polymorphic sites, 3 or 4 putative monomorphic elements are present which may explain the more modest differential up-regulation of the luciferase expression by GATA4 and YY1 over-expression in the context of the two haplotypes (≈1.6 fold) compared to their differential binding capacity indicated in the EMSAs (at least 3-5 fold).

The observation that GATA4 is the predominant GATA member bound on FCGR2B promoter in B lymphocytes and monocytes is surprising. Of the 6 known GATA family members, GATA1, GATA2, and GATA3 are expressed predominantly in hematopoietic cells, while GATA4, GATA5 and GATA6 are predominantly expressed in the developing heart and several endodermal lineages (68). Mutations in GATA4 have recently been shown to cause human congenital heart defects (69). However, the gene-specific RT-PCR utilized in these studies for GATA1, 2, 3, 4, 5, and 6 demonstrates that GATA4 is the major GATA expressed in B lymphoid BJAB cells and myeloid U937 cells. These results are in agreement with previous findings that GATA4 are expressed in primary monocytes by western blot analysis and immunohistochemistry (70). Little is known about GATA expression in B lymphocytes, and western blot analysis using anti-GATA1 and GATA2 antibodies suggest that neither of these family members is expressed in BJAB cells. Gene-specific RT-PCR in EBV-transformed B cells and primary tonsil cells detected GATA3 and GATA4 messages, supporting GATA4 expression in B cells. Interestingly, synergistic effects of GATA and YY1 on gene transcription have been reported. Human FcERI α-chain gene expression is synergistically upregulated by GATA1 and YY1 (71). The cardiac B-type natriuretic peptide promoter is cooperatively activated by GATA4 and YY1 (33). This synergistic effect also exists for FCGR2B gene in the context of 2B.4 (−386C-120A) haplotype.

The 2B.4 FCGR2B promoter haplotype leads to increased receptor expression on both B lymphocytes and monocytes. Donors with heterozygous haplotypes have 1.5-fold elevated receptor expression compared to donors with homozygous common haplotype on EBV-transformed and fresh peripheral B-lymphocytes. One donor with homozygous variant haplotype has 2.5-fold increased receptor expression on EBV-cells. Similar differential FcγRIIb expression is seen on CD14⁺ monocytes. Thus, it may be reasonable to speculate that on dendritic cells, derived from either lymphoid or myeloid lineages, the levels of FcγRIIb are regulated by similar mechanisms and that these promoter haplotypes will lead to similar differences among individuals.

The role of FcγRIIb in autoimmunity may be more intricate than that of a “negative” regulator since it subserves multiple functions on different cell types. For example, on mononuclear phagocytes, FcγRIIb can decrease the uptake and clearance of immune complexes (7), which might prolong circulation of autoantigens, increase availability of such antigens for processing at other sites, and enhance the tissue deposition of immune complexes with subsequent tissue injury. On follicular dendritic cells (FDC), FcγRIIb promotes the maturation of FDC reticulum and may enhance the antibody recall responses of memory B cells (10-13, 49). FcγRIIb also mediates antigen internalization and presentation on other dendritic cell types (58, 59, 73). From these perspectives, a relative increase in FcγRIIb expression and function might decrease the clearance of antigenic, apoptotic material by macrophages and increase DC-mediated processing and presentation of these autoantigens.

On B cells, FcγRIIb plays an important regulatory role in BCR signaling and antibody production. Co-engagement of FcγRIIb by IgG complexes down-modulates B cell activation and provides a negative feedback mechanism for IgG production. One can speculate that FcγRIIb polymorphisms play a role in the regulation of antibody responses, and indeed, low-expression FcγRIIb polymorphisms may lead to higher humoral immune responses in mouse (56). Complete deficiency of FcγRIIb, combined with a permissive genetic background, leads to an autoimmune phenotype. In humans, however, no FcγRIIb deficiency has been identified yet, and the expression levels of FcγRIIb in SLE patients are complicated by the disease stages and activity. The genetic association studies presented herein suggest that the gain-of-function FcγRIIb polymorphisms are enriched in SLE patients compared to ethnically matched controls. Perhaps, altered FcγRIIb expression may influence negative selection occurring in immature B-lymphocytes in bone marrow and in transitional B cells in the peripheral lymphoid organs. These two stages of negative selection are critical in maintaining immune tolerance to self-antigens. Although largely unexplored, FcγRIIb negatively regulates pre-BCR-mediated signaling for apoptosis in the pre-B cell stage (62). In the cell viability assays provided herein, FcγRIIb negatively regulates IgM BCR-induced decrease in cell viability in EBV-transformed peripheral blood B cells. Although this observation may reflect a combination of effects on cell proliferation and cell death, apoptosis-specific Annexin V and activated caspase-3 staining suggested that apoptosis did occur during this process. Thus, elevated FcγRIIb expression may provide a mechanism for leakiness in the negative selection of autoreactive B cells. Indeed, it has been shown that estrogen and prolactin promote autoimmunity by altering thresholds for B cell apoptosis and rescuing the autoreactive B cells that would normally be deleted (74-76). Over-expression of the anti-apoptotic Bc1-2 in several transgenic mouse models enhances survival of the bone marrow-derived autoreactive B cells and of autoreactive B cells that arise in germinal centers following somatic mutation (77-79). Thus in considering modulation of FcγRIIb expression as a therapeutic target, it may be important to consider cell-type specific targeting according to the disease stage and characteristics. It may be that the predominant, pathophysiologically important cell type to target will vary according to the nature and stage of the autoimmune process.

Taken together, the data presented herein characterized the two FCGR2B promoter haplotypes which affect endogenous receptor expression on primary B lymphocytes and monocytes. The association of the high expression haplotype with human SLE suggests the contribution of FcγRIIb to autoimmune susceptibility and implicates that cell-type specific modulation of FcγRIIb expression with consideration of specific disease pathogenesis may be important in the treatment of autoimmune diseases. Furthermore, FCGR2B promoter genotypes can also play an important role in the variations of human responses to vaccines and to antibody-based therapeutic drugs.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Daeron, M. 1997. Fc receptor biology. Annu Rev Immunol 15:203.
2. Ravetch, J. V., and S. Bolland. 2001. IgG Fc receptors. Annu Rev Immunol 19:275.
3. Takai, T. 2002. Roles of Fc receptors in autoimmunity. Nat Rev Immunol 2:580.
4. Coggeshall, K. M. 2002. Regulation of signal transduction by the Fc gamma receptor family members and their involvement in autoimmunity. Curr Dir Autoimmun 5:1.
5. Vivier, E., and M. Daeron. 1997. Immunoreceptor tyrosine-based inhibition motifs. Immunol Today 18:286.
6. Bolland, S., and J. V. Ravetch. 1999. Inhibitory pathways triggered by ITIM-containing receptors. Adv Immunol 72:149.
7. Pricop, L., P. Redecha, J. L. Teillaud, J. Frey, W. H. Fridman, C. Sautes-Fridman, and J. E. Salmon. 2001. Differential modulation of stimulatory and inhibitory Fcg receptors on human monocytes by Th1 and Th2 cytokines. J Immunol 166:531.
8. Tridandapani, S., K. Siefker, J. L. Teillaud, J. E. Carter, M. D. Wewers, and C. L. Anderson. 2002. Regulated expression and inhibitory function of FcgRIIb in human monocytic cells. J Biol Chem 277:5082.
9. Hunter, S., Z. K. Indik, M. K. Kim, M. D. Cauley, J. G. Park, and A. D. Schreiber. 1998. Inhibition of Fcg receptor-mediated phagocytosis by a nonphagocytic Fcg receptor. Blood 91:1762.
10. Qin, D., J. Wu, K. A. Vora, J. V. Ravetch, A. K. Szakal, T. Manser, and J. G. Tew. 2000. Fcg receptor IIB on follicular dendritic cells regulates the B cell recall response. J Immunol 164:6268.
11. Tew, J. G., J. Wu, M. Fakher, A. K. Szakal, and D. Qin. 2001. Follicular dendritic cells: beyond the necessity of T-cell help. Trends Immunol 22:361.
12. Haberman, A. M., and M. J. Shlomchik. 2003. Reassessing the function of immune-complex retention by follicular dendritic cells. Nat Rev Immunol 3:757.
13. Kosco-Vilbois, M. H. 2003. Are follicular dendritic cells really good for nothing? Nat Rev Immunol 3:764.
14. Jiang, Y., S. Hirose, M. Abe, R. Sanokawa-Akakura, M. Ohtsuji, X. Mi, N. Li, Y.

Xiu, D. Zhang, J. Shirai, Y. Hamano, H. Fujii, and T. Shirai. 2000. Polymorphisms in IgG Fc receptor IIB regulatory regions associated with autoimmune susceptibility. Immunogenetics 51:429.
15. Jiang, Y., S. Hirose, R. Sanokawa-Akakura, M. Abe, X. Mi, N. Li, Y. Miura, J. Shirai, D. Zhang, Y. Hamano, and T. Shirai. 1999. Genetically determined aberrant down-regulation of FcgRIIB1 in germinal center B cells associated with hyper-IgG and IgG autoantibodies in murine systemic lupus erythematosus. Int Immunol 11:1685.
16. Pritchard, N. R., A. J. Cutler, S. Uribe, S. J. Chadban, B. J. Morley, and K. G. Smith. 2000. Autoimmune-prone mice share a promoter haplotype associated with reduced expression and function of the Fc receptor FcgRII. Curr Biol 10:227.
17. Bolland, S., Y. S. Yim, K. Tus, E. K. Wakeland, and J. V. Ravetch. 2002. Genetic modifiers of systemic lupus erythematosus in FcgRIIB(−/−) mice. J Exp Med 195:1167.
18. Nakamura, A., T. Yuasa, A. Ujike, M. Ono, T. Nukiwa, J. V. Ravetch, and T. Takai. 2000. Fcg receptor IIB-deficient mice develop Goodpasture's syndrome upon immunization with type IV collagen: a novel murine model for autoimmune glomerular basement membrane disease. J Exp Med 191:899.
19. Kleinau, S., P. Martinsson, and B. Heyman. 2000. Induction and suppression of collagen-induced arthritis is dependent on distinct Fcg receptors. J Exp Med 191:1611.
20. Takai, T., M. Ono, M. Hikida, H. Ohmori, and J. V. Ravetch. 1996. Augmented humoral and anaphylactic responses in FcgRII-deficient mice. Nature 379:346.
21. Bolland, S., and J. V. Ravetch. 2000. Spontaneous autoimmune disease in FcgRIIB-deficient mice results from strain-specific epistasis. Immunity 13:277.
22. Tsokos, G. C. 2001. Systemic lupus erythematosus. A disease with a complex pathogenesis. Lancet 358 Suppl:S65.
23. Shlomchik, M. J., J. E. Craft, and M. J. Mamula. 2001. From T to B and back again: positive feedback in systemic autoimmune disease. Nat Rev Immunol 1:147.
24. Khan, I. U., G. C. Tsokos, and G. M. Kammer. 2003. Abnormal B cell signal transduction in systemic lupus erythematosus. Curr Dir Autoimmun 6:89.
25. Davidson, A., and B. Diamond. 2001. Autoimmune diseases. N Engl J Med 345:340.
26. Wakeland, E. K., K. Liu, R. R. Graham, and T. W. Behrens. 2001. Delineating the genetic basis of systemic lupus erythematosus. Immunity 15:397.
27. Wanstrat, A., and E. Wakeland. 2001. The genetics of complex autoimmune diseases: non-MHC susceptibility genes. Nat Immunol 2:802.
28. Salmon, J. E., S. Millard, L. A. Schachter, F. C. Arnett, E. M. Ginzler, M. F. Gourley, R. Ramsey-Goldman, M. G. Peterson, and R. P. Kimberly. 1996. FcgRIIA alleles are heritable risk factors for lupus nephritis in African Americans. J Clin Invest 97:1348.
29. Wu, J., J. C. Edberg, P. B. Redecha, V. Bansal, P. M. Guyre, K. Coleman, J. E. Salmon, and R. P. Kimberly. 1997. A novel polymorphism of FcgRIIIa (CD 16) alters receptor function and predisposes to autoimmune disease. J Clin Invest 100:1059.
30. Seligman, V. A., C. Suarez, R. Lum, S. E. Inda, D. Lin, H. Li, J. L. Olson, M. F. Seldin, and L. A. Criswell. 2001. The Fcg receptor IIIA-158F allele is a major risk factor for the development of lupus nephritis among Caucasians but not non-Caucasians. Arthritis Rheum 44:618.
31. Dijstelbloem, H. M., M. Bijl, R. Fijnheer, R. H. Scheepers, W. W. Oost, M. D. Jansen, W. J. Sluiter, P. C. Limburg, R. H. Derksen, J. G. van de Winkel, and C. G. Kallenberg. 2000. Fcg receptor polymorphisms in systemic lupus erythematosus: association with disease and in vivo clearance of immune complexes. Arthritis Rheum 43:2793.
32. Edberg, J. C., C. D. Langefeld, J. Wu, K. L. Moser, K. M. Kaufman, J. Kelly, V. Bansal, W. M. Brown, J. E. Salmon, S. S. Rich, J. B. Harley, and R. P. Kimberly. 2002. Genetic linkage and association of Fcg receptor IIIA (CD16A) on chromosome 1q23 with human systemic lupus erythematosus. Arthritis Rheum 46:2132.
33. Dijstelbloem, H. M., J. G. van de Winkel, and C. G. Kallenberg. 2001. Inflammation in autoimmunity: receptors for IgG revisited. Trends Immunol 22:510.
34. Nishimura, H., M. Nose, H. Hiai, N. Minato, and T. Honjo. 1999. Development of lupus-like autoimmune diseases by disruption of the PD-1 gene encoding an ITIM motif-carrying immunoreceptor. Immunity 11:141.
35. Prokunina, L., C. Castillejo-Lopez, F. Oberg, I. Gunnarsson, L. Berg, V. Magnusson, A. J. Brookes, D. Tentler, H. Kristjansdottir, G. Grondal, A. I. Bolstad, E. Svenungsson, I. Lundberg, G. Sturfelt, A. Jonssen, L. Truedsson, G. Lima, J. Alcocer-Varela, R. Jonsson, U. B. Gyllensten, J. B. Harley, D. Alarcon-Segovia, K. Steinsson, and M. E. Alarcon-Riquelme. 2002. A regulatory polymorphism in PDCD1 is associated with susceptibility to systemic lupus erythematosus in humans. Nat Genet 32:666.
36. Warmerdam, P. A., J. G. van de Winkel, A. Vlug, N. A. Westerdaal, and P. J. Capel. 1991. A single amino acid in the second Ig-like domain of the human Fc gamma receptor II is critical for human IgG2 binding. J Immunol 147:1338.
37. Cooper, G. S., M. A. Dooley, E. L. Treadwell, E. W. St Clair, and G. S. Gilkeson. 2002. Risk factors for development of systemic lupus erythematosus: allergies, infections, and family history. J Clin Epidemiol 55:982.
38. Lewontin, R. C. 1964. The interaction of selection and linkage. I. General considerations: Heterotic models. Genetics 49:49.
39. Li, X., J. Wu, R. H. Carter, J. C. Edberg, K. Su, G. S. Cooper, and R. P. Kimberly. 2003. A novel polymorphism in the Fcgamma receptor IIB (CD32B) transmembrane region alters receptor signaling. Arthritis Rheum 48:3242.
40. Warmerdam, P. A., N. M. Nabben, S. A. van de Graaf, J. G. van de Winkel, and P. J. Capel. 1993. The human low affinity immunoglobulin G Fc receptor IIC gene is a result of an unequal crossover event. J Biol Chem 268:7346.
41. Qiu, W. Q., D. de Bruin, B. H. Brownstein, R. Pearse, and J. V. Ravetch. 1990. Organization of the human and mouse low-affinity FcgR genes: duplication and recombination. Science 248:732.
42. Su, K., J. Wu, J. C. Edberg, S. E. McKenzie, and R. P. Kimberly. 2002. Genomic organization of classical human low-affinity Fcg receptor genes. Genes Immun 3:S51.
43. Cameron, A. J., K. J. McDonald, M. M. Harnett, and J. M. Allen. 2002. Differentiation of the human monocyte cell line, U937, with dibutyryl cyclicAMP induces the expression of the inhibitory Fc receptor, FcgRIIb. Immunol Lett 83:171.
44. Estienne, V., C. Duthoit, M. Reichert, A. Praetor, P. Carayon, W. Hunziker, and J. Ruf. 2002. Androgen-dependent expression of FcgRIIB2 by thyrocytes from patients with autoimmune Graves' disease: a possible molecular clue for sex dependence of autoimmune disease. Faseb J 16:1087.
45. Rudge, E. U., A. J. Cutler, N. R. Pritchard, and K. G. Smith. 2002. Interleukin 4 reduces expression of inhibitory receptors on B cells and abolishes CD22 and FcgRII-mediated B cell suppression. J Exp Med 195:1079.
46. Su, K., X. Li, J. C. Edberg, J. Wu, P. Ferguson, and R. P. Kimberly. 2004. A promoter haplotype of the ITIM-bearing FcgRIIb alters receptor expression and associates with autoimmunity. II. Differential binding of GATA4 and YY1 transcription factors and correlated receptor expression and function. J. Immunol. In press.
47. Kyogoku, C., H. M. Dijstelbloem, N. Tsuchiya, Y. Hatta, H. Kato, A. Yamaguchi, T. Fukazawa, M. D. Jansen, H. Hashimoto, J. G. van de Winkel, C. G. Kallenberg, and K. Tokunaga. 2002. Fcg receptor gene polymorphisms in Japanese patients with systemic lupus erythematosus: contribution of FCGR2B to genetic susceptibility. Arthritis Rheum 46:1242.
48. Gabriel, S. B., S. F. Schaffner, H. Nguyen, J. M. Moore, J. Roy, B. Blumenstiel, J. Higgins, M. DeFelice, A. Lochner, M. Faggart, S. N. Liu-Cordero, C. Rotimi, A. Adeyemo, R. Cooper, R. Ward, E. S. Lander, M. J. Daly, and D. Altshuler. 2002. The structure of haplotype blocks in the human genome. Science 296:2225.
49. Rao, S. P., K. A. Vora, and T. Manser. 2002. Differential expression of the inhibitory IgG Fc receptor FcgRIIB on germinal center cells: implications for selection of high-affinity B cells. J Immunol 169:1859.
50. Tsao, B. P., R. M. Cantor, and F. M. Arnett. 2000. Investigation of SLE-linked regions identified by genome scans: support for 1q23, 16q13 and 20p12. Arthritis Rheum 43:S278.
51. Moser, K. L., B. R. Neas, J. E. Salmon, H. Yu, C. Gray-McGuire, N. Asundi, G. R. Bruner, J. Fox, J. Kelly, S. Henshall, D. Bacino, M. Dietz, R. Hogue, G. Koelsch, L. Nightingale, T. Shaver, N. I. Abdou, D. A. Albert, C. Carson, M. Petri, E. L. Treadwell, J. A. James, and J. B. Harley. 1998. Genome scan of human systemic lupus erythematosus: evidence for linkage on chromosome 1q in African-American pedigrees. Proc Natl Acad Sci USA 95:14869.
52. Johanneson, B., G. Lima, J. Von Salome, D. Alarcon-Segovia, and M. E. Alarcon-Riquelme. 2002. A major susceptibility locus for systemic lupus erythemathosus maps to chromosome 1q31. Am J Hum Genet 71:1060.
53. Shai, R., F. P. Quismorio, L. Lily, O. Kwon, J. Morrison, and D. J. Wallace. 1999. Genome-wide screen for systemic lupus erythematosus susceptibility genes in multiplex families. Hum Mol Genet 8:639.
54. Ueda, H., J. M. Howson, L. Esposito, J. Heward, H. Snook, G. Chamberlain, D. B. Rainbow, K. M. Hunter, A. N. Smith, G. Di Genova, M. H. Herr, I. Dahlman, F. Payne, D. Smyth, C. Lowe, R. C. Twells, S. Howlett, B. Healy, S. Nutland, H. E. Rance, V. Everett, L. J. Smink, A. C. Lam, H. J. Cordell, N. M. Walker, C. Bordin, J. Hulme, C. Motzo, F. Cucca, J. F. Hess, M. L. Metzker, J. Rogers, S. Gregory, A. Allahabadia, R. Nithiyananthan, E. Tuomilehto-Wolf, J. Tuomilehto, P. Bingley, K. M. Gillespie, D. E. Undlien, K. S. Ronningen, C. Guja, C. Ionescu-Tirgoviste, D. A. Savage, A. P. Maxwell, D. J. Carson, C. C. Patterson, J. A. Franklyn, D. G. Clayton, L. B. Peterson, L. S. Wicker, J. A. Todd, and S. C. Gough. 2003. Association of the T-cell regulatory gene CTLA4 with susceptibility to autoimmune disease. Nature 423:506.
55. Bailey, J. A., Z. Gu, R. A. Clark, K. Reinert, R. V. Samonte, S. Schwartz, M. D. Adams, E. W. Myers, P. W. Li, and E. E. Eichler. 2002. Recent segmental duplications in the human genome. Science 297:1003.
56. Xiu, Y., K. Nakamura, M. Abe, N. Li, X. S. Wen, Y. Jiang, D. Zhang, H. Tsurui, S. Matsuoka, Y. Hamano, H. Fujii, M. Ono, T. Takai, T. Shimokawa, C. Ra, T. Shirai, and S. Hirose. 2002. Transcriptional Regulation of Fcgr2b Gene by Polymorphic Promoter Region and Its Contribution to Humoral Immune Responses. J Immunol 169:4340.
57. Hulett, M. D., and P. M. Hogarth. 1994. Molecular basis of Fc receptor function. Adv Immunol 57:1.
58. Esposito-Farese, M. E., C. Sautes, H. de la Salle, S. Latour, T. Bieber, C. de la Salle, P. Ohlmann, W. H. Fridman, J. P. Cazenave, J. L. Teillaud, and et al. 1995. Membrane and soluble FcgRII/III modulate the antigen-presenting capacity of murine dendritic epidermal Langerhans cells for IgG-complexed antigens. J Immunol 155:1725.
59. Amigorena, S. 2002. Fc gamma receptors and cross-presentation in dendritic cells. J Exp Med 195:F1.
60. Guermonprez, P., J. Valladeau, L. Zitvogel, C. Thery, and S. Amigorena. 2002. Antigen presentation and T cell stimulation by dendritic cells. Annu Rev Immunol 20:621.
61. Pearse, R. N., T. Kawabe, S. Bolland, R. Guinamard, T. Kurosaki, and J. V. Ravetch. 1999. SHIP recruitment attenuates FcgRIIB-induced B cell apoptosis. Immunity 10:753.
62. Kato, I., T. Takai, and A. Kudo. 2002. The pre-B cell receptor signaling for apoptosis is negatively regulated by FcgRIIB. J Immunol 168:629.
63. Su, K., J. Wu, J. C. Edberg, X. Li, P. Ferguson, G. S. Cooper, C. D. Langefeld, and R. P. Kimberly. 2004. A promoter haplotype of the ITIM-bearing FcgRIIb alters receptor expression and associates with autoimmunity. I. Regulatory FCGR2B polymorphisms and their association with systemic lupus erythematosus. J Immunol. in revision.
64. Trainor, C. D., J. G. Omichinski, T. L. Vandergon, A. M. Gronenborn, G. M. Clore, and G. Felsenfeld. 1996. A palindromic regulatory site within vertebrate GATA-1 promoters requires both zinc fingers of the GATA-1 DNA-binding domain for high-affinity interaction. Mol Cell Biol 16:2238.
65. Jacobsen, B. M., and D. G. Skalnik. 1999. YY1 binds five cis-elements and trans-activates the myeloid cell-restricted gp91(phox) promoter. J Biol Chem 274:29984.
66. Cassel, D. L., M. A. Keller, S. Surrey, E. Schwartz, A. D. Schreiber, E. F. Rappaport, and S. E. McKenzie. 1993. Differential expression of FcgRIIA, FcgRIIB and FcgRIIC in hematopoietic cells: analysis of transcripts. Mol Immunol 30:451.
67. Sato, S., M. Hasegawa, M. Fujimoto, T. F. Tedder, and K. Takehara. 2000. Quantitative genetic variation in CD19 expression correlates with autoimmunity. J Immunol 165:6635.
68. Patient, R. K., and J. D. McGhee. 2002. The GATA family (vertebrates and invertebrates). Curr Opin Genet Dev 12:416.
69. Garg, V., I. S. Kathiriya, R. Barnes, M. K. Schluterman, I. N. King, C. A. Butler, C. R. Rothrock, R. S. Eapen, K. Hirayama-Yamada, K. Joo, R. Matsuoka, J. C. Cohen, and D. Srivastava. 2003. GATA4 mutations cause human congenital heart defects and reveal an interaction with TBX5. Nature 424:443.
70. Caramori, G., S. Lim, K. Ito, K. Tomita, T. Oates, E. Jazrawi, K. F. Chung, P. J. Barnes, and I. M. Adcock. 2001. Expression of GATA family of transcription factors in T-cells, monocytes and bronchial biopsies. Eur Respir J 18:466.
71. Nishiyama, C., M. Hasegawa, M. Nishiyama, K. Takahashi, Y. Akizawa, T. Yokota, K. Okumura, H. Ogawa, and C. Ra. 2002. Regulation of human Fc epsilon RI alpha-chain gene expression by multiple transcription factors. J Immunol 168:4546.
72. Bhalla, S. S., L. Robitaille, and M. Nemer. 2001. Cooperative activation by GATA-4 and YY1 of the cardiac B-type natriuretic peptide promoter. J Biol Chem 276:11439.
73. Yada, A., S. Ebihara, K. Matsumura, S. Endo, T. Maeda, A. Nakamura, K. Akiyama, S. Aiba, and T. Takai. 2003. Accelerated antigen presentation and elicitation of humoral response in vivo by FcgammaRIIB- and FcgammaRI/III-mediated immune complex uptake. Cell Immunol 225:21.
74. Bynoe, M. S., C. M. Grimaldi, and B. Diamond. 2000. Estrogen up-regulates Bc1-2 and blocks tolerance induction of naive B cells. Proc Natl Acad Sci USA 97:2703.
75. Grimaldi, C. M., J. Cleary, A. S. Dagtas, D. Moussai, and B. Diamond. 2002. Estrogen alters thresholds for B cell apoptosis and activation. J Clin Invest 109:1625.
76. Peeva, E., D. Michael, J. Cleary, J. Rice, X. Chen, and B. Diamond. 2003. Prolactin modulates the naive B cell repertoire. J Clin Invest 111:275.
77. Hande, S., E. Notidis, and T. Manser. 1998. Bc1-2 obstructs negative selection of autoreactive, hypermutated antibody V regions during memory B cell development. Immunity 8:189.
78. Hartley, S. B., M. P. Cooke, D. A. Fulcher, A. W. Harris, S. Cory, A. Basten, and C. C. Goodnow. 1993. Elimination of self-reactive B lymphocytes proceeds in two stages: arrested development and cell death. Cell 72:325.
79. Smith, K. G., A. Light, L. A. O'Reilly, S. M. Ang, A. Strasser, and D. Tarlinton. 2000. bc1-2 transgene expression inhibits apoptosis in the germinal center and reveals differences in the selection of memory B cells and bone marrow antibody-forming cells. J Exp Med 191:475.

What is claimed is:

1. A nucleic acid comprising an FCGR2B promoter comprising SEQ ID NO:1, wherein SEQ ID NO: 1 comprises one or more polymorphic sites.

2. The nucleic acid of claim 1, wherein one or more polymorphic sites are selected from the group consisting of a polymorphism at position −120, a polymorphism at position −386, a polymorphism at position −893, a polymorphism at position −1153, a polymorphism at position −1223, a polymorphism as position −1443, a polymorphism at position −1614, a polymorphism at position −1700, a polymorphism at position −1867 and a polymorphism at position −1868.

3. A method of characterizing a FCGR2B gene comprising the step of identifying nucleotides at one or more polymorphic sites in the promoter nucleic acid, the identified nucleotides indicating the character of the polymorphic FCGR2B gene.

4. The method of claim 3, wherein the polymorphic site is at position −386 of the promoter.

5. The method of claim 4, wherein the polymorphic site at position −386 contains a C or a G at this position.

6. The method of claim 3, wherein the polymorphic site is at position −120 of the promoter.

7. The method of claim 6, wherein the polymorphic site at position −120 contains an A or a T at this position.

8. The method of claim 3, wherein the polymorphic sites are at positions −120 and −386 of the promoter.

9. The method of claim 8, wherein the polymorphic site at position −120 contains an A or a T at this position and wherein the polymorphic site at position −386 contains a C or a G at this position.

10. The method of claim 3, wherein the step of identifying the nucleotide at the polymorphic site or sites comprises comparing the promoter sequence to a reference promoter sequence.

11. The method of claim 3, wherein the identifying step comprises obtaining a biological sample and testing the sample to identify the nucleotide at the polymorphic site in the nucleic acid contained therein.

12. The method of claim 11, wherein the sample is tested by sequencing or probing the nucleic acid.

13. The method of claim 11, wherein the testing step comprises the step of amplifying the nucleic acid contained in the sample.

14. The method of claim 13, wherein the testing step further comprises sequencing the amplified nucleic acid.

15. The method of claim 13, wherein the amplifying step comprises a polymerase chain reaction (PCR).

16. The method of claim 15, wherein the amplifying step comprises contacting the nucleic acid with a primer comprising the sequence of

| | |
|---|---|
| AAAGAGGGTGGAAAGGGAGGAG | (SEQ ID NO: 21) |
| or | |
| CTCTCAAAGCTTGGCGGATTCTAC. | (SEQ ID NO: 22) |

17. The method of claim 15, wherein the amplifying step comprises contacting the nucleic acid with a primer comprising the sequence of

| | |
|---|---|
| TCAAGAAGCATCCAGAT | (SEQ ID NO: 23) |
| or | |
| AAACTCAGCTCAGAACCTCCTGTT. | (SEQ ID NO: 24) |

18. A method for determining a FCGR2B promoter haplotype in a human subject comprising identifying a nucleotide present at a one or more polymorphic sites in either or both copies of the promoter contained in the subject's genomic nucleic acids, wherein the nucleotide present at the polymorphic site or sites indicates the promoter haplotype.

19. The method of claim 18, wherein the identifying step comprises identifying the nucleotide at position −120, at position −386, or both.

20. The method of claim 18, wherein the haplotype is selected from the group consisting of −386C/−120A, −386G/−120T, −386G/−120A. −386C/−120T.

21. A method of determining a subject's predisposition to an inflammatory disease comprising comparing the subject's FCGR2B promoter haplotype with one or more reference promoter haplotypes that correlate with elevated FcγRIIb levels, a similar haplotype in the subject's FCGR2B promoter as compared to the reference promoter haplotype or haplotypes indicating a predisposition to the inflammatory disease.

22. The method of claim 21, wherein the inflammatory disease is an autoimmune disease.

23. The method of claim 21, further comprising comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating a predisposition to the inflammatory disease.

24. The method of claim 21, further comprising comparing the subject's FCGR2B transmembrane domain with one or more reference polymorphic transmembrane domains that correlate with increased FcγRIIb activity, a similar transmembrane domain as compared to the reference transmembrane domains further indicating a predisposition to the inflammatory disease.

25. The method of claim 21, further comprising comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating a predisposition to the inflammatory disease.

26. The method of claim 21, further comprising two or more of the following:
a. comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating a predisposition to the inflammatory disease.
b. comparing the subject's FCGR2B transmembrane domain with one or more reference polymorphic transmembrane domains that correlate with increased FcγRIIb activity, a similar transmembrane domain as compared to the reference transmembrane domains further indicating a predisposition to the inflammatory disease; and
c. comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating a predisposition to the inflammatory disease.

27. A method of determining a subject's susceptibility to an infection comprising comparing the subject's FCGR2B promoter haplotype with one or more reference promoter haplotypes that correlate with reduced FcγRIIb levels, a similar haplotype in the subject's FCGR2B promoter as compared to the reference promoter haplotype or haplotypes indicating a subject's susceptibility to infection.

28. The method of claim 27, further comprising comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating a subject's susceptibility to infection.

29. The method of claim 27, further comprising comparing the subject's FCGR2B transmembrane domain with one or more reference polymorphic transmembrane domains that correlate with increased FcγRIIb activity, a similar transmembrane domain as compared to the reference transmembrane domains further indicating a subject's susceptibility to infection.

30. The method of claim 27, further comprising comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating a subject's susceptibility to infection.

31. The method of claim 27, further comprising two or more of the following:
a. comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating a subject's susceptibility to infection
b. comparing the subject's FCGR2B transmembrane domain with one or more reference polymorphic transmembrane domains that correlate with increased FcγRIIb activity, a similar transmembrane domain as compared to the reference transmembrane domains further indicating a subject's susceptibility to infection; and
c. comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating a subject's susceptibility to infection.

32. A method of determining a subject's ability to mount an immune response comprising comparing the subject's FCGR2B promoter haplotype with one or more reference promoter haplotypes that correlate with elevated FcγRIIb levels, a similar haplotype in the subject's FCGR2B promoter as compared to the reference promoter haplotype or haplotypes indicating the subject's ability to mount an immune response.

33. The method of claim 32, further comprising comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating the subject's ability to mount an immune response.

34. The method of claim 32, further comprising comparing the subject's FCGR2B transmembrane domain with one or more reference polymorphic transmembrane domains that correlate with increased FcγRIIb activity, a similar transmembrane domain as compared to the reference transmembrane domains further indicating the subject's ability to mount an immune response.

35. The method of claim 32, further comprising comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating the subject's ability to mount an immune response.

36. The method of claim 32, further comprising two or more of the following:
a. comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating the subject's ability to mount an immune response;
b. comparing the subject's FCGR2B transmembrane domain with one or more reference polymorphic transmembrane domains that correlate with increased FcγRIIb activity, a similar transmembrane domain as compared to the reference transmembrane domains further indicating the subject's ability to mount an immune response; and
c. comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating the subject's ability to mount an immune response.

37. A method of determining a subject's responsiveness to an immunoglobulin based therapeutic agent comprising comparing the subject's FCGR2B promoter haplotype with one or more reference promoter haplotypes that correlate with modulated FcγRIIb levels, a similar haplotype in the subject's FCGR2B promoter as compared to the reference promoter haplotype or haplotypes indicating the subject's responsiveness to an immunoglobulin based therapeutic agent.

38. The method of claim 37, further comprising comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating the subject's responsiveness to an immunoglobulin based therapeutic agent.

39. The method of claim 37, further comprising comparing the subject's FCGR2B transmembrane domain with one or more reference polymorphic transmembrane domains that correlate with increased FcγRIIb activity, a similar transmembrane domain as compared to the reference transmembrane domains further indicating the subject's responsiveness to an immunoglobulin based therapeutic agent.

40. The method of claim 37, further comprising comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating the subject's responsiveness to an immunoglobulin based therapeutic agent.

41. The method of claim 37, further comprising two or more of the following:
a. comparing the subject's FCGR3A extracellular domain with one or more reference extracellular domain polymorphic sequences that correlate with reduced FcγRIIIa activity, a similar extracellular domain in the subject's FCGR3A extracellular domain as compared to the reference extracellular domain sequences further indicating the subject's responsiveness to an immunoglobulin based therapeutic agent;
b. comparing the subject's FCGR2B transmembrane domain with one or more reference polymorphic transmembrane domains that correlate with increased FcγRIIb activity, a similar transmembrane domain as compared to the reference transmembrane domains further indicating the subject's responsiveness to an immunoglobulin based therapeutic agent; and
c. further comprising comparing the subject's FCGR3A cytoplasmic domain with one or more reference polymorphic FCGR3A cytoplasmic domains that correlate with reduced FcγRIIIa activity, a similar cytoplasmic domain as compared to the reference cytoplasmic domains further indicating the subject's responsiveness to an immunoglobulin based therapeutic agent

42. A method for determining a FCGR2B promoter haplotype in a population of human subjects comprising identifying a nucleotide present at a one or more polymorphic sites in either or both copies of the promoter contained in the subjects' genome, wherein the nucleotide present at the polymorphic site or sites indicates the promoter haplotype of each subject.

43. A method of selecting a population of human subjects for a treatment with a immunoglobulin based therapeutic agent comprising the steps of
a. determining each potential subject's responsiveness to the immunoglobulin based therapeutic agent according to the method of any of claims 37-41;
b. selecting those subjects with responsiveness to the immunoglobulin based therapeutic agent.

44. A method of selecting treatment for a disorder in a subject, comprising the steps of
a. determining a FCGR2B promoter haplotype in the subject according to the method of claim 18; and
b. selecting the treatment based on the FCGR2B promoter haplotype.

45. The method of claim 44, wherein the disorder is an inflammatory disease.

46. The method of claim 45, wherein the inflammatory disease is an autoimmune disease.

47. The method of claim 46, wherein treatment is selected to decrease FcγRIIb activity or to increase FcγRIIIa activity.

48. A method of identifying a compound that modulates FcγRIIb levels comprising:
a. contacting with a test compound a cell containing a FCGR2B promoter nucleic acid sequence comprising selected nucleotides at one or more polymorphic sites at residues −386 and −120 in the FCGR2B promoter, wherein the promoter nucleic acid sequence is operatively linked to a nucleic acid sequence encoding a reporter protein;
b. detecting the amount of reporter protein expressed by the cell after contact with the test compound; and
c. comparing the amount of reporter protein in the contacted cell with the amount of reporter protein in a control cell, wherein the control cell is not contacted by the test compound, an increased or decreased amount of reporter protein in the test cell as compared to the control cell indicating a compound that modulates FcγRIIb levels.

49. A method of selecting a therapy for a subject, the method comprising:
a. comparing the FCGR2B promoter haplotype of the subject to a plurality of reference FCGR2B promoter haplotypes, wherein each reference FCGR2B promoter haplotype has a value, each value corresponding to a selected therapy; and
b. selecting the reference FCGR2B promoter haplotype most similar to the subject's FCGR2B promoter haplotype, to thereby select a therapy for the subject.

50. A computer system comprising:
a. a database including records comprising a plurality of reference haplotypes comprising the SNPs of Table 1 and associated diagnosis and therapy data; and
b. a user interface capable of receiving a selection of one or more test haplotypes for use in determining matches between the test haplotypes and the reference haplotypes and displaying the records associated with matching haplotypes.
